(19) United States
(12) Patent Application Publication
(10) Pub. No.: US 2017/0091406 A1
(43) Pub. Date: Mar. 30, 2017

(54) SYSTEM AND METHOD FOR MANAGING ILLNESS OUTSIDE OF A HOSPITAL ENVIRONMENT

(71) Applicant: Eqol Inc., Sault Ste. Marie (CA)

(72) Inventors: Binh Tranh Nguyen, Toronto (CA); Jonathan Mark Tomkun, Waterloo (CA)

(73) Assignee: Eqol Inc., Sault Ste. Marie, ON (CA)

(21) Appl. No.: 15/126,813

(22) PCT Filed: Mar. 17, 2015
(86) PCT No.: PCT/CA2015/000164

§ 371 Date: Sep. 16, 2016

Related U.S. Application Data
(60) Provisional application No. 61/954,048, filed on Mar. 17, 2014.

(51) Int. Cl. G06F 19/00; G06Q 10/08

(52) U.S. Cl. CPC G06F 19/3418; G06Q 10/087; G06F 19/325; G06F 19/3493

(57) ABSTRACT

Systems and methods for managing illness outside of a hospital environment are provided. A plurality of alert generation definitions is stored with each of the definitions defining at least one condition for generating an alert. A patient profile data structure for a patient is generated which includes indicators of characteristics of the patient. Monitoring data is received, which reflects at least one of (i) physiological indicators of the patient; and (ii) indicators of medical supplies being ordered or consumed by the patient. An alert relating to patient healthcare is generated by applying at least one of the alert generation definitions to the received monitoring data. Population data reflective of healthcare outcomes for a patient population is received. At least one further alert generation definition is generated upon processing the population data, and the at least one further alert generation definition is stored.

### FIELD OF THE INVENTION

This invention relates generally to health care management systems, and more particularly to systems and methods for managing illness outside of a hospital environment.

### BACKGROUND OF THE INVENTION

There is an ever-growing demand on health care systems. As a result, many clinical procedures, especially related to chronic disease management, are being shifted from the hospital into the home.

The shift from the hospital to the home not only saves costs for the health care system but also provides many potential advantages to the patient such as increased independence and health outcome. However, this change is often met by resistance from patients who feel safer in the monitored, regimented environment of the hospital. There is considerable fear and apprehension associated with independent or home procedures.

The fear and apprehension is linked with the perceived lack of monitoring and communication with health care providers, reduced access to resources, and an overwhelming number of tasks to manage when performing procedures independently.

In addition to the challenges faced by patients, there are also inefficiencies in the workflow of the health care team (HCT).

In the current framework, clinical documentation is a valuable but overlooked component of the treatment process. Data relating to activity, progress, and inventory is often collected by the patients themselves using pen and paper. The data is often only made available to the HCT when the patient brings the physical logs to the hospital or when the HCT visits the patient's home. This data is then reported to health administrators on a monthly or quarterly basis. For example, many patients who are in need of care for issues related to renal failure, such as patients on dialysis, record much of their data on paper, their data being the state of their renal failure, the filtration rate associated with their dialysis, etc.

This process is suboptimal and results in inefficiency, poor data integrity, and low data resolution and accuracy.

Conventional monitoring systems enable clinicians to care for their patients remotely. However, due to geographical separation, there are limits to how much clinicians are able to do. In addition, there are high costs associated with all tasks, regardless of complexity, as the tasks revolve around the clinician. This model creates a very high demand on health care resources.

Electronic Medical Record (EMR) technology attempts to consolidate clinical documentation. However, typical EMRs receive input directly from healthcare professionals and the problem of an increased demand on healthcare resources persists.

Competing solutions include monitoring systems and patient card systems.

Monitoring systems are costly and card systems are extremely limited in functionality.

A new solution is thus needed for overcoming the shortfalls of the solutions currently available in the market.

### SUMMARY OF THE INVENTION

The present disclosure relates to a system and method for managing illness outside of a hospital environment.

In an aspect, a system for monitoring healthcare of patients who are outside of a hospital environment is provided, the system comprising: an electronic alert data storage for storing a plurality of alert generation definitions, each of the alert generation definitions defining at least one condition for generating an alert; a patient profile module configured to generate a patient profile data structure for a patient, the patient profile data structure comprising indicators of characteristics of the patient; a patient monitoring module configured to: receive monitoring data reflecting at least one of (i) physiological indicators of the patient; and (ii) indicators of medical supplies being ordered or consumed by the patient; and generate an alert relating to patient healthcare by applying at least one of the plurality of alert generation definitions to the received monitoring data; and an alert update module configured to: receive population data reflective of healthcare outcomes for a patient population and monitoring data for the patient population; generate at least one further alert generation definition upon processing the population data; and update the electronic alert data storage with the at least one further alert generation definition.

In another aspect, the population data further comprises data reflective of generated alerts for the patient population.

In another aspect, generating at least one further alert generation definition comprises training an artificial neural network using the population data.

In another aspect, the system further includes an alert notification module configured to present a notification of the generated alert to a healthcare provider.

In another aspect, the system further includes: a network interface configured for interconnection with a communication network; and wherein at least part of the monitoring data is received by way of the communication network.

In another aspect, at least part of the monitoring data is received from one or more medical devices.

In another aspect, a machine learning technique is used to generate at least one further alert generation definition upon processing the population data.

In another aspect, the machine learning technique includes the use of trained neural networks.

In another aspect, the alert update module is configured to generate the at least one further alert generation definition upon processing the population data by: provisioning a neural network configured to refine the alert generation definitions for an alert using the population data; applying one or more weights corresponding each of the at least one conditions for generating the alert, wherein an aggregate of the weights is used in determining whether an alert is triggered; generating one or more predicted outcome based on the alert rule; providing the population data as an input into the neural network, the population data having various inputs and actual outcomes; causing the neural network to iteratively process alert generation definitions based on the input population data, wherein the one or more weights corresponding to each of the at least one condition are varied, and on each iteration: using population data related to an individual in a population, determine whether each of the at least one conditions for generating the alert corresponds to a related actual outcome, if so, increase the weight corresponding to that condition, and if not, decrease the weight corresponding to that condition; generating a further alert generation definition based on the one or more weights corresponding to each of the at least one condition following the use of the neural network to process iterations of alert generation definitions; and generating a further alert generation definition based on the one or more weights corresponding to each of the at least one condition following the use of the neural network to process iterations of alert generation definitions.

In another aspect, the population data includes indicators of medical supplies being ordered or consumed by other patients.

In another aspect, the further alert generation definition is based on processed population data including at least both physiological indicators of other patients and indicators of medical supplies being ordered or consumed by other patients.

In a further aspect, a computer-implemented method of monitoring healthcare of patients who are outside of a hospital environment is provided. The method includes: storing a plurality of alert generation definitions in electronic alert data storage, each of the alert generation definitions defining at least one condition for generating an alert; generating, at at least one processor, a patient profile data structure for a patient, the patient profile data structure comprising indicators of characteristics of the patient; receiving, at the at least one processor, monitoring data reflecting at least one of (i) physiological indicators of the patient; and (ii) indicators of medical supplies being ordered or consumed by the patient; and generating, at the at least one processor, an alert relating to patient healthcare by applying at least one of the plurality of alert generation definitions to the received monitoring data; receiving, at the at least one processor, population data reflective of healthcare outcomes for a patient population and monitoring data for the patient population; generating, at the at least one processor, at least one further alert generation definition upon processing the population data; and updating, at the at least one processor, the electronic alert data storage with the at least one further alert generation definition.

In another aspect, the method may further include receiving, at the at least one processor, further monitoring data; and generating, at the at least one processor, a further alert relating to patient healthcare by applying the at least one further alert generation definition to the received further monitoring data.

In yet another aspect, a system for facilitating management of chronic conditions in patient residences is provided. The system includes: a patient profile module configured to generate a patient profile data structure for a patient, the patient profile data structure comprising: indicators of a chronic condition of the patient, and indicators of a treatment for the patient; a patient monitoring module configured to receive monitoring data reflecting at least one of (i) physiological indicators of the patient; and (ii) indicators of medical supplies being ordered or consumed by the patient; and a decision support module configured to: process the patient profile data structure and the received monitoring data; upon said processing, generate treatment suggestions for the patient; and present the generated treatment suggestions to the patient.

In another aspect, the decision support module is configured to: upon said processing, provide an analysis of the processed data to a health care provider.

In another aspect, the system includes a communications module configured to:
provide a communication interface for interaction between the patient and a health care provider.

In another aspect, the communications module is configured to match the patient with other patients for interaction therewith, the matching comprising processing at least one of the (i) the patient profile data structure and (ii) the received monitoring data; and provide a communication interface for interaction between the patient and the other patients.

In another aspect, the chronic condition of the patient includes renal failure.

In another aspect, the medical supplies being ordered or consumed by the patient include at least one of face masks, gauze, bandages, dialysate, catheters, needles, and dialysate bags.

In another aspect, the indicators of medical supplies being ordered or consumed by the patient are provided by one or more suppliers of the medical supplies.

In another aspect, the indicators of medical supplies being ordered or consumed by the patient include logistics information related to the delivery of supplies to a patient's residence.

In another aspect, the logistics information includes at least one of frequency of delivery, expected time of delivery, reliability of delivery, and lead-time for placing a supply order.

In a yet further aspect, a computer-implemented method for facilitating management of chronic conditions in patient residences is provided. The method includes: generating, at at least one processor, a patient profile data structure for a patient, the patient profile data structure comprising: indicators of a chronic condition of the patient, and indicators of a treatment for the patient; receiving, at the at least one processor, monitoring data reflecting at least one of (i) physiological indicators of the patient; and (ii) indicators of medical supplies being ordered or consumed by the patient; processing, at the at least one processor, the patient profile data structure and the received monitoring data; upon said processing, generating, at the at least one processor, a treatment suggestion for the patient; and presenting the generated treatment suggestion to the patient.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

In the drawings, embodiments of the invention are illustrated by way of example. It is to be expressly understood that the description and drawings are only for the purpose of illustration and as an aid to understanding, and are not intended as a definition of the limits of the invention.

FIG. 1 is an illustrative diagram providing a generic computer hardware and software implementation of certain aspects, as detailed in the description.

FIG. 2 is a block diagram providing an illustration of a simplified embodiment of the invention.

FIG. 3 is a block diagram illustrating a backend system, according to some embodiments of the invention.

FIG. 4 is an inventory ordering workflow, according to some embodiments of the invention.

FIG. 5 is an example patient treatment workflow, according to some embodiments of the invention.

FIG. 6 is an example patient treatment workflow, according to some embodiments of the invention.

FIG. 7 is an example patient treatment workflow, according to some embodiments of the invention.

FIG. 8 is an example workflow for using the support portal, according to some embodiments of the invention.

FIG. 9 is an example inventory workflow, according to some embodiments.

FIG. 10 provides a sample workflow for the generation of alerts, according to some embodiments.

FIG. 11 is a sample screenshot of an interface for entering patient profile data, according to some embodiments.

FIG. 12 is a sample screenshot of an interface providing a patient the ability to self-record information following treatment, according to some embodiments

FIG. 13 is a sample screenshot of an interface providing a patient the ability to log blood pressure information, according to some embodiments.

FIG. 14 is a sample screenshot of an interface providing dashboard information for a patient, according to some embodiments.

FIG. 15 is a sample screenshot of an interface for confirming supply counts, according to some embodiments.

FIG. 16 is a sample screenshot of an interface for preparing an order for shipment to the patient, according to some embodiments

FIG. 17 is a sample screenshot for an interface providing a dashboard view of information relating to the care of a patient, according to some embodiments,

FIG. 18 is a sample screenshot of a patient's blood pressure level charted against time, according to some embodiments.

FIG. 19 is a sample screenshot of an interface configured for providing information related to alerts, according to some embodiments.

FIG. 20 is a sample workflow illustrating steps that may be performed in updating an electronic alert data storage with further alert generation definitions based on healthcare outcomes for a patient population, according to some embodiments.

FIG. 21 provides a sample workflow for the generation of treatment suggestions to a patient, according to some embodiments.

FIG. 22 is an example workflow of the generation of alerts using a neural network, according to some embodiments.

### DETAILED DESCRIPTION

The present invention, in one aspect, provides a system for managing and monitoring treatment for patients outside of a hospital environment.

The system may include:
a. at least one module configured to provide an interface for interacting with one or more patients;
b. at least one module configured to provide an interface for interacting with one or more clinicians; and
c. at least one module configured to develop and apply a set of rules to one or more data sets received from the one or more patients or the one or more clinicians.

In an aspect, the system is a patient-centered solution to help individuals manage their chronic conditions in their residences. The system may include the features of conventional monitoring systems, such that doctors may be able to monitor their patients and intervene whenever it is necessary to do so. In addition, the system may incorporate patient-oriented tools to enable the one or more patients to respond to non-urgent issues. The system may also contain patient training and reference media, and potentially allowing administrative users to customize the training and reference media.

In an aspect, the system provides tools for process and inventory management, such as a syncing messaging system and calendar to the patient that allows them to receive messages or events from their clinician with proper notifications and reminders.

In an some aspects of the invention, the system is designed to work with existing EMR systems that may already be in place at different institutions, interacting with these systems at varying levels of integration ranging from simply pulling data from external EMRs to both pushing and pulling data. The system may also be able to operate as its own standalone medical record.

In an aspect, the system may provide tools for enabling clinical documentation by the patient, remote customization of patient settings and features, up-to-date inventory tracking and management assistance, dynamic analysis of current data, machine learning in determining patient difficulties, and instructing patients in the leading practice procedures for resolving difficulties, if available.

In an aspect, the system may provide tools for patients to interact with one another, for example, using the consumption of the supplies as a basis to match the patient to other patients at a similar stage or have a similar health condition to share insights.

In an aspect, information regarding supplies including inventory levels, placed orders, usage rates, invoices, etc. may be exchanged with industry suppliers to assist them in helping patients access supplies necessary for their homecare.

The stakeholders for this system include: one or more patients, one or more care providers, one or more administrators and one or more industry partners.

Industry partners may include suppliers of health care products and inventory; manufacturers and retailers of health care machines, etc.

According to an embodiment of the invention, the system is comprised of an interface that dialysis patients interact with, as well as a web interface that clinicians interact with. These interfaces may be implemented as mobile applications or desktop applications. Patients may input data into the patient interface before and following each treatment. The data is then made accessible to clinicians and other members of the patient's care team through the support interface. The support interface may provide individualized access requiring authorization to each clinician (doctor/nurse), technologist, and administrator through a secure login system. The individualized access may provide different users with a layout and functionality specific to their needs and responsibilities within the healthcare ecosystem. The patient interface may also educate and guide the patient through dialysis using various learning media. The platform may interface with various medical devices (e.g. weight scales, blood pressure monitors, glucometers, etc.) via a multitude of current and legacy connection protocols including Bluetooth, Bluetooth low energy, USB, serial, infrared, NFC, WiFi, ANT+, etc. The interface, in some embodiments of the invention, may also be implemented using appropriate application programming interfaces (APIs).

In some aspects of the invention, the focus is the renal illnesses and dialysis, providing a system to help patients and their care teams with the transition into dialysis and the continued management of dialysis treatment. In these aspects of the invention, the system may facilitate communication, training, clinical documentation, inventory management and scheduling between patients, clinicians, and administration.

In alternate aspects of the invention, the features and functionality are provided to support and optimize processes for all forms of independent care or care that is facilitated outside of the hospital environment. For example, this system may be used to monitor post-operative recovery, prescription drug adherence, rehabilitation, diet, diabetes, cancer therapies, respiratory conditions, heart disease, mental illness, among others.

Potential advantages to using the system may include better adherence to treatment and clinical prescriptions, which may result in improved patient health outcomes and improved efficiency of administrating treatment for both patients and caregivers, saving time and money for those involved with patient care.

The systems and methods may conduct various tasks in monitoring health care of patients who are outside of a hospital environment, and in some embodiments, these tasks may be grouped broadly into the following example categories:
1. The generation of a patient-specific monitoring profile from information gathered about the patient from various sources (e.g., dialysis machine, supply reordering, electronic health record, self-reported information). This information may comprise indicators of various characteristics of a patient (e.g., age, date of birth, treatment stage, symptoms, blood pressure).
2. Medical supply analysis (e.g., a cost analysis) for generating predictions and identifying problems (e.g., by comparing the individual to aggregate herd information, identifying and/or matching correlations).
3. Generation of warnings and alerts based on applied rules (e.g., to an individual, a physician, a caregiver, an insurance company).
4. Alert rule template generation using various machine learning techniques (e.g., artificial neural networks, trained to process patient information (e.g., new information, historical information, population-level information, interrelationships), and identify one or more relationships (e.g., a marker indicative for increased/decreased costs, increased/decreased health outcomes, increased/decreased prevalence of side effects, dialysis machine performance issues).

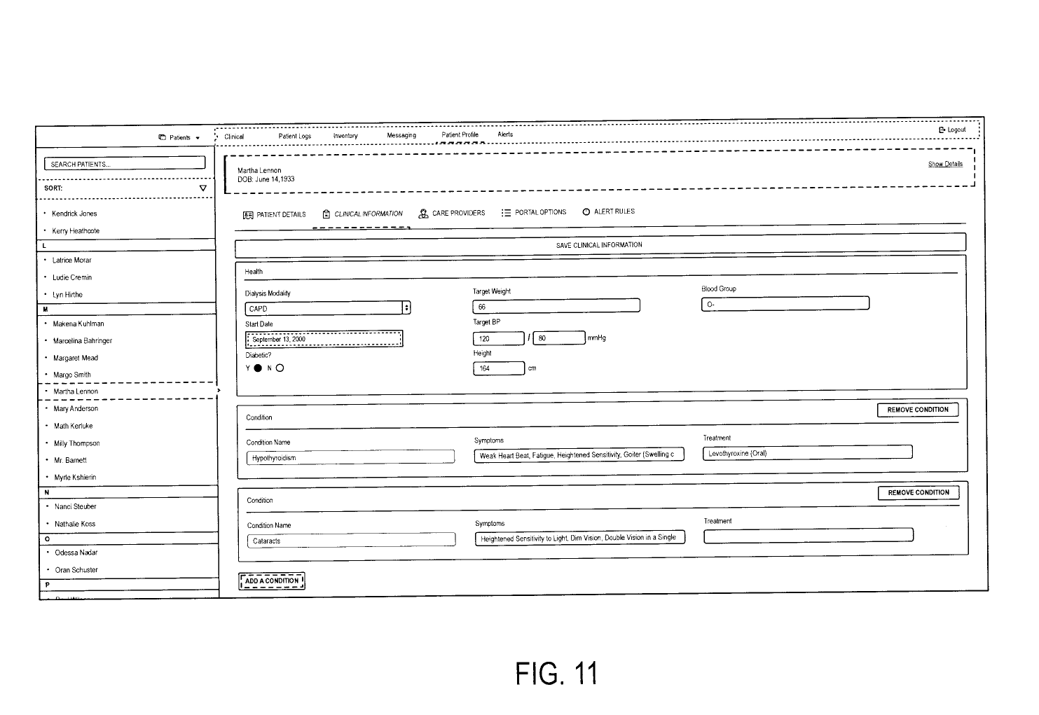

FIG. 11 is a sample screenshot of an interface for entering patient profile data, according to some embodiments. Various elements of information may be captured about a patient, such as date of birth, identity of care providers, patient details, clinical information, treatment information (e.g., dialysis modality, start date, diabetes status, target weight target blood pressure, blood group, height), and conditions (e.g., condition name, symptoms, treatment). This information may also be provided from electronic medical records, hospital records, etc.

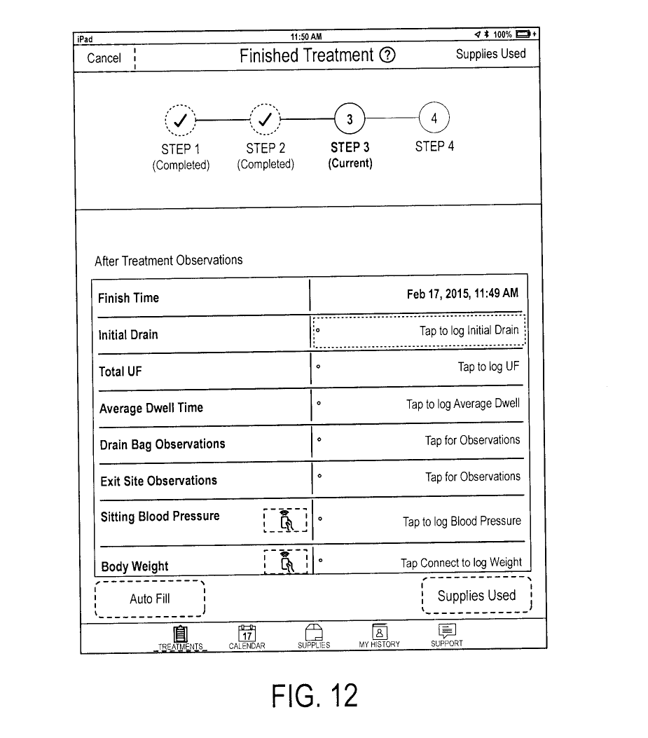

FIG. 12 is a sample screenshot of an interface providing a patient the ability to self-record information following treatment, according to some embodiments. In the context of dialysis treatment, the patient may, for example, record information such as finish time, initial drain, total ultrafiltration, average dwell time, drain bag observations, exit site observations, blood pressure, body weight, supplies used, etc. There may be, for example, information capture points at the various steps of a treatment, such as beginning a new treatment, following up after a treatment, etc. This information may also be provided directly from information captured by one or more medical devices (e.g., a dialysis machine).

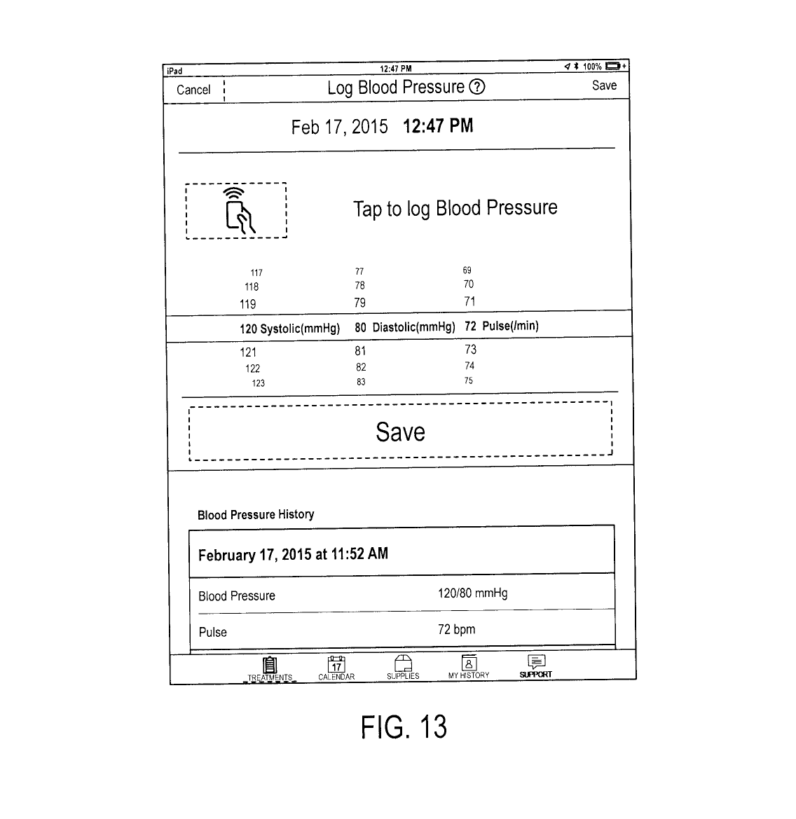

FIG. 13 is a sample screenshot of an interface providing a patient the ability to log blood pressure information, according to some embodiments. Other interfaces may be provided, for example, to permit information to be received and/or logged relating to blood sugar, weight, supply counts, etc. This information may also be provided from automated and/or semi-automated sources, such as biometric tracking devices, web-enabled medical devices, etc.

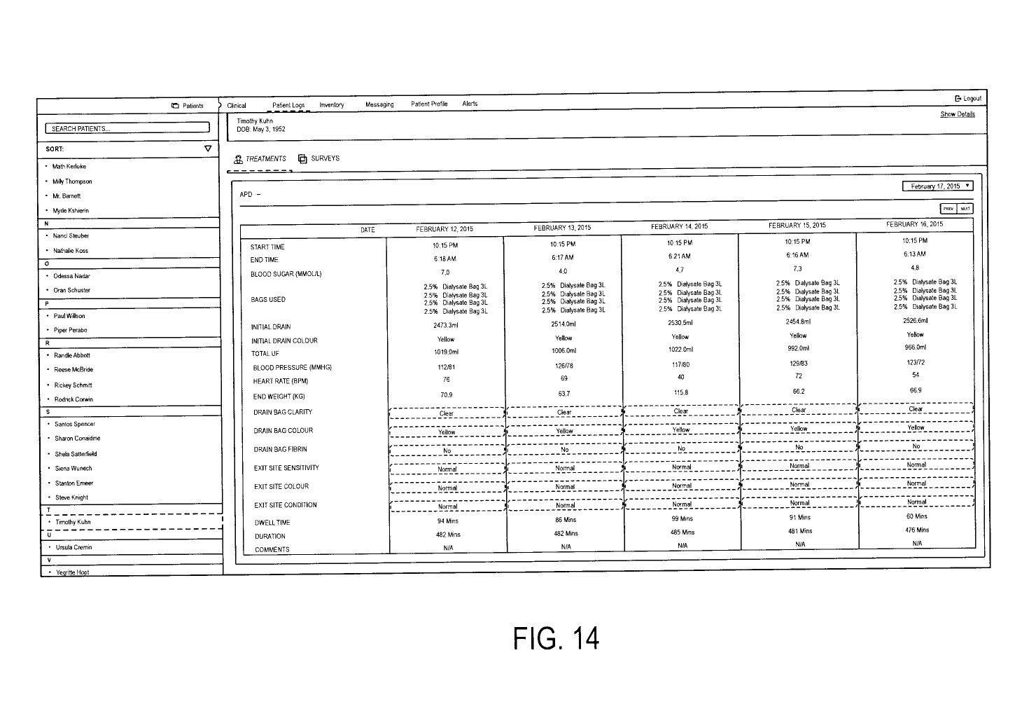

FIG. 14 is a sample screenshot of an interface providing dashboard information for a patient, according to some embodiments. Information may be provided, such as the date of treatment and information associated with the treatment provided each day. The dashboard may be used to, view, for example, messages communicated with healthcare practitioners, logs, alerts, etc.

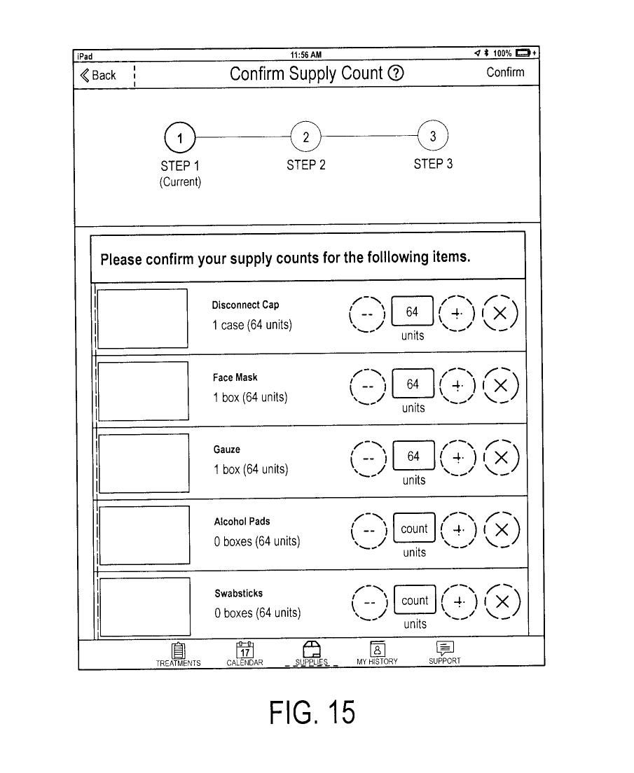

FIG. 15 is a sample screenshot of an interface for confirming supply counts, according to some embodiments. The interface can be used for keeping track of the number of units for various medical supplies, such as disconnect caps, face masks, gauze, alcohol pads, swab-sticks, etc. This information may be received from other sources, such as supplier information, logistics portals, etc.

Steps related to each category may be taken individually or in combination by the system, and inputs and/or outputs from one or more steps may be used as inputs and/or outputs from other steps.

One or more logical rules may be applied to conduct various determinations and/or to generate various alerts. The logical rules, for example, may be set such that an action is triggered when an unsafe condition is detected (e.g., deteriorating kidney function, low blood pressure, electrolyte abnormality, infection/sepsis), incorrect usage is detected (e.g., too many/not enough bandages being used, low/high rate of solution consumption), among others.

There may be interactions with external systems (e.g., through various interfaces), patient information and data sources, including sensory information and/or device information.

Various algorithms, machine learning techniques, trend prediction, vector machines and artificial neural networks may be used in applying, tuning and/or refining the various logical rules. For example, the data may be interpolated, predictions may be generated, relationships identified, etc.

In some embodiments, population-level information (“population data”) may be used to train an artificial neural network. The training may include, for example, iterative approaches to analyze quantities of information for various individuals, the application of adaptive weighing and/or transformation functions, etc. The training may include supervised learning, unsupervised learning, reinforcement learning techniques, etc. This training may be used, for example, to refine one or more conditions that define when an alert should be triggered. The training may use the population data to iteratively determine when conditions corresponded with a particular outcome associated with the condition in relation to actual outcomes experienced by other patients. Conditions more closely correlated to outcomes may be assigned higher weights, and conditions less closely correlated to outcomes may be assigned lower weights, the weights being adjusted across training iterations.

Some potential advantages of using a trained artificial neural network include the ability to conduct open-ended analysis, the ability to identify otherwise difficult to find relationships, the ability to be used in relation to a highly complex set of inputs and outputs, etc. For example, it may be assumed according to conventional knowledge that a condition leads to an outcome, but through an analysis of a large set of population data, it may be revealed that the condition is only loosely related to the outcome, and perhaps another condition may be the primary cause.

The trained artificial neural network may be continually and/or frequently refined based on incoming information, as it relates to the population level, or is specific to one or more individuals. As the data set grows (e.g., as provided or received over time), the accuracy and/or ability of the trained artificial neural network may increase. In some embodiments, the population data used as input into the neural network is a subset of the full set of population data, selecting only population data for patients who have one or more similar characteristics to a particular patient (e.g., only males or only females).

In some embodiments, the trained artificial neural network may be provided inputs that include one or both patient physiological data and supply data. For example, dialysis is a supply-intensive care practice, and the use of supplies may provide a proxy indicator for other characteristics of care being provided to a patient.

For example, a patient may be using tubing at a high rate, which may be indicative of hygiene or bacterial infection problems; or a patient could be using dialysis pressure bandages at a high rate, which may be indicative of issues with post-dialysis wound healing.

In some embodiments, the system may be configured to generate one or more supply predictions by applying various machine learning techniques to patient supply use, quantizing and predicting trends, aggregating these trends and/or constructing overall supply reporting at a population level, and then identifying problems in individuals by comparison to the herd and specific similar subsets derived from the portions or all of the population (the “herd”).

In some embodiments, the system may be configured to generate one or more cost predictions by comparing patient clinical measurements to averages calculated in the population and determining correlations to a patient's cost of treatment and clinical outcomes. These outputs may be used, for example, by practitioners to support decision making regarding current and future budget allocations. The prediction process may involve attaching a support vector machine to an artificial neural network.

The support vector machine may be trained on existing data and values may be assigned to the neural network's neurons in order to predict the next set of data. Predictions are compared against existing data. After each iteration, the support vector machine may be configured to modify the weigh associated with pathways to improve prediction accuracy based on the error margins of a previous prediction. The various pathways and weights may be captured in one or more functions having various weighted conditions as inputs, the aggregate weights of satisfied conditions being used to determine when a function should trigger an alert.

The iteration process may be conducted until the prediction is accurate within a given margin (e.g., within a predefined threshold). The artificial neural network receives its input from the support vector machine, and produces an output, which is a refined prediction. The iteration process may be conducted continually as new data (e.g., population data) becomes available.

In some embodiments, the artificial neural network is used to iterate and develop functions for the estimation of the future cost of clinical outcomes (e.g., supply cost, healthcare costs).

For example, when a function with a high degree of accuracy is produced (e.g., 50-70 year old patients with highly variable weight may lead to increased cost due to hospitalization due to supply misuse), the identified weightings and the function conditions are utilized to generate a new alert rules template that may be applied to various patients.

When the conditions of the function are triggered for a patient, an alert may be provided to the clinical team. Conditions may vary, including determining that a patient issues an excessive order from an iPad™, that a patient logs weight from outside of a targeted weight zone, etc.

In some embodiments, the system may be configured to generate one or more alerts such as clinical warnings, based on one or more alert rules (alert generation definitions) generated using machine learning and regression techniques on the population. For example, these alerts may be used by practitioners to catch errors and problems much earlier based on previous errors and problems that may not be readily apparent to the human user.

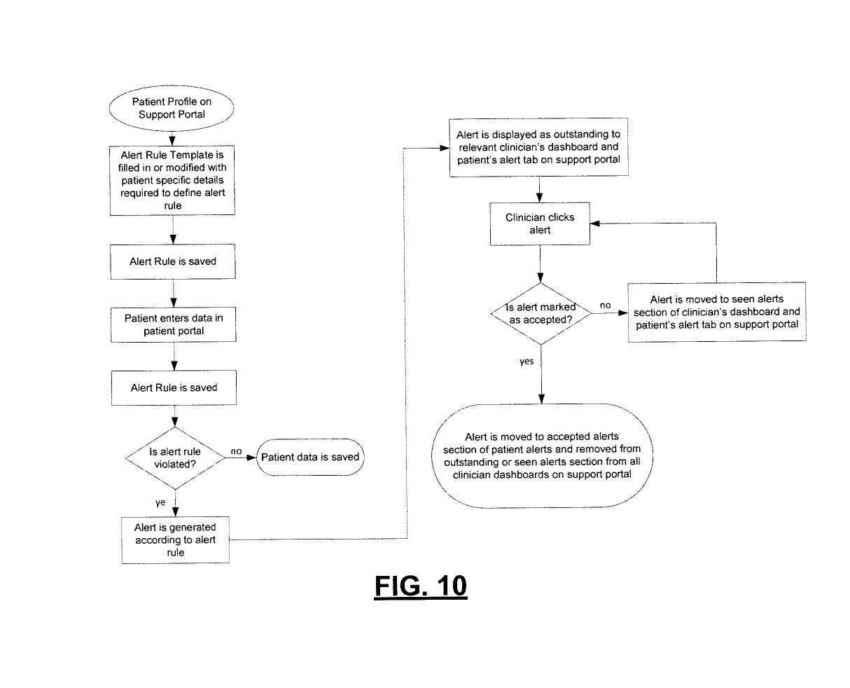

FIG. 10 provides a sample workflow for the generation of alerts that may be generated based on various alert rules, according to some embodiments. As provided in the workflow, alert rule templates may be adapted based on information provided by a patient profile or other information, creating an alert rule. The alert rule may be configured for updating on a continuous or other basis, for example, when new information is provided by the patient or related to the patient. Where an alert rule is violated, a clinician may be alerted, for example, through a dashboard, and the alert can be marked as accepted and/or seen, depending on the clinician's interactions with the alert.

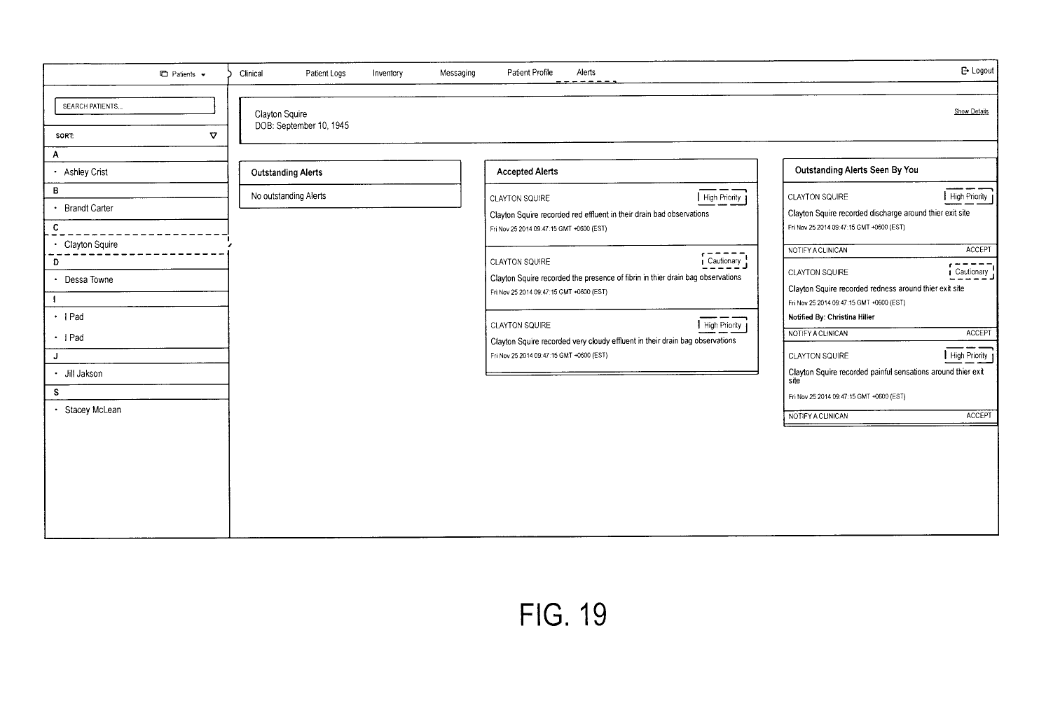

FIG. 19 is a sample screenshot of an interface configured for providing information related to alerts, according to some embodiments. As depicted, the alerts may be grouped based on various criteria, such as by status (e.g., outstanding, accepted and outstanding alerts).

For example, in one specific embodiment, new alert rules may be created for individual patients using the following schema whose definitions are a Chomsky Type 0 (Recursively Enumerable) language based loosely on the LISP programming language.

```
            value
                  a number
            literal (type of value)
                  A literal is a number given as an immediate to an
                  instruction.
                  A type of value
                  Numbers should be integers or floats
                  ex. (add 1 expression) - here 1 is a literal
            expression -> value
                  an expression is anything which can be evaluated to
                  a value.
            variable -> expression
                  a variable name. Must be defined in the variable
field to be reference in the rule field since it evaluates to an expression,
variables must have a value.
            Conditional -> (if <test_expression>
            <if_true_expression> <else expression>)
            procedure -> <function definition>
                  (lambda (<param1>...<paramN>) <expression>)
                  Function to be called with the parameters 1 to N.
```

For example an alert for a high diastolic by reading may be:

```
            (>(getattr BloodPressure diastolic @3){{ }})
          The language for the above schema may be defined as follows:
          tokenization ->
          value(token) ->
            Evaluates a single token to a machine compatible
representation. For example, the value of “1” in a program string is
evaluated to the integer 1 for internal representation.
          expression ->
            <value>
            OR
            (<procedure>) <parameter-expression 1> <parameter-
            expression 2> ...)
            OR
            (<built-in procedure> <parameter-expression-1>
            <parameter expression 2> ... )
            An expression is evaluated to an internal-representation of a
value by being recursively evaluated until a value is found.
```

Sample built in procedures include the + function, so an example expression may include:
(+(+3 5) 4)

which when parsed into types, may be provided as:
(built-in-procedure (built-in-procedure value value) value)
and evaluates to add(add(3,5), 4)->12, where add is a function which adds its two parameters.

procedure->(lambda (<param 1><param 2> . . . )<expression>)
(lambda (<param1><paramN>)<expression>)

The above may be used to define a function which can then be called with the parameters 1 to N.

The grammar for this language is tokenized by matched pairs of parenthesis to define expressions and spaces between other tokens, in the style of the LISP programming language.

The above example provides that if a patient's blood pressure is above a certain value 3 consecutive times, an alert is generated. The rules can be modified from patient to patient depending on the patient's personal circumstances. The system may be configured for the assembling of new expressions to catch and generate alerts related to problems that may not have been encountered previously. Thresholds and other conditions for generating alerts may be adjusted over time, e.g., based on new population data or as the patient's profile changes (e.g., as other aspects of the patient's health condition changes). Such conditions may be adjusted by way of one or more of the machine-learning processes disclosed herein.

Clinical and/or other physiological information may be collected for the generation and/or updating of a patient ‘profile’. Inputs may include, for example, patient basic physiological data (as provided from records, from practitioners (nurses, doctors, clinicians, etc.), self-reported information or other information available to clinical team). Information may include body weight, target blood pressure, age/date of birth, etc.

In some embodiments, physiological information is received from one or more interfaces with dialysis machines. For example, data may automatically be retrieved from one or more sensors or processors associated with a dialysis machine, tracking information such as dialysate usage, dialysis rate, characteristics of removed fluids, operating times, error messages, power consumption, blood and/or fluid flow rates, potential infection information, maintenance information (line flushing), etc.

In some embodiments, other information may be retrieved from suppliers and/or provider of supplies, such as hospitals, clinics, etc. This information, for example, may indicate the types of products used, the frequency in which they have been provided, the current amount of product remaining, etc. Medical supplies may include, for example, bandages, dialysate, dialysate bags, medical masks, gloves, tubing, catheters, antibiotics, hormones, and saline solutions.

The information may be saved to a persistent electronic data store (e.g., providing a database) for use by the system.

Supply analysis may utilize inputs such as stored clinical information, data provided by a patient (e.g., through a patient portal, recording daily blood pressure, daily supply use), etc. Supply analysis may include, for example, a determination of the total costs for patient in a time period. This step may be also be performed at a population level or for one or more groups and/or subgroups of patients to identify population level statistics and to provide for one or more reference points to compare datasets and outputs. Patterns, averages, relationships, etc. may be identified.

Following an analysis for a particular patient, the system may be configured to run various reports to identify for example, the most expensive (e.g., the top 10%) patients, the least expensive (e.g., the bottom 10%) patients, and any identified trend. For example, where there are no extenuating causes for high costs incurred by a particular patient relative to the population level, it may be indicative that patients costs can be reduced.

Various healthcare outcomes may be tracked, such as fever, abdominal pain, cloudy effluent, vomiting, swollen appendages, slow inflow of dialysate, renal failure, and slow outflow of dialysate, among others.

Alerts may be generated based on data from patients. These alerts may be provided in the form of notifications, text messages, calls, emails, etc. The alerts may be provided to suppliers, practitioners, patients, etc. For example, the alerts may be specific for a particular supply order and may be based on particular characteristics of the supply order, such as information about the frequency in which the supplies are provided to one or more patients.

Alerts may be triggered based on the applications of various rules—the rules may have various default settings and may also be modified by clinicians for specific patients (e.g., if a patient logs 3 weights which are inside their ‘heavy’ range as recorded in the Clinical Information Step, generate an alert).

Alerts may be logged to a support portal, sent via text, email, pager, etc.

In some embodiments, there may be various automated or semi-automated processes configured for the generation of alert rule templates. For example, various machine-learning processes may be used to define various attributes of rules, and refine these rules over a period of time as more data is introduced.

In some embodiments, input and/or predicted/actual output information may be analyzed to determine various linkages. As described above, one or more neural networks may be trained to generate alert rules and/or templates based on various predicted linkages that may be captured in the forms of logical rules that may be refined over a period of time.

For example, an artificial neural network may be trained by a support vector machine to process new patient information and may be configured to provide a function that identifies patient records or sets of patient records as a marker for increased costs or changes in clinical outcomes.

In some cases, a manual, automated and/or semi-automated process may review the rule as generated by the neural network and if judged applicable, the logical rule may be adapted as a template for one or more alert rules. These alert rule templates, for example, may be used to determine patient overdosing, over ordering, under-ordering, etc., and may also be used for comparisons between information known about individual patients and population level information, predicted vs. actual outcomes, etc.

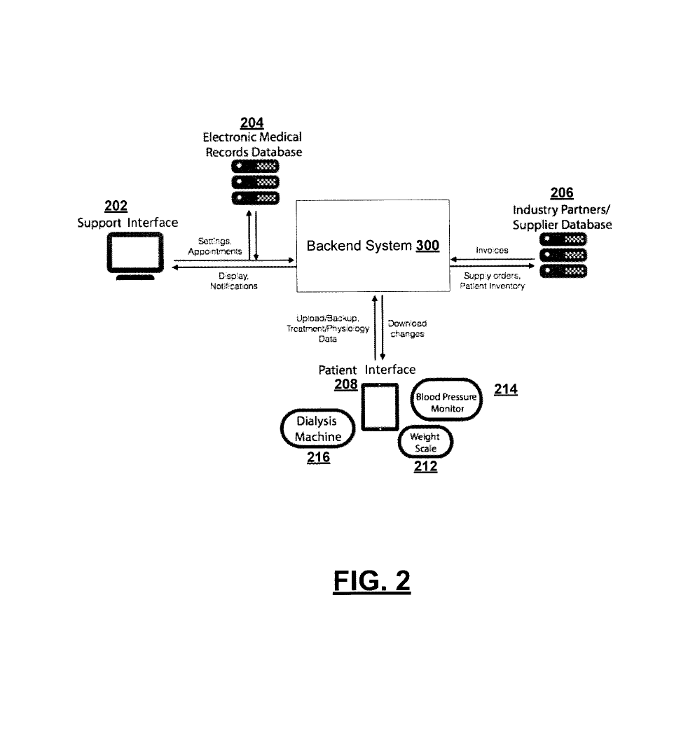

Referring now to FIG. 2, FIG. 2 is a schematic diagram providing an illustration of a simplified embodiment of the invention.

The backend system 300 is connected to a support interface 202, one or more electronic medical records databases 204, one or more industry partners/supplier databases 206, a patient interface 208.

The patient interface 208 and the support interface 202 may be accessible via suitable network technology over one or more networks 250. The one or more networks 250 may include the internet, intranets, point to point networks, etc. Networking technology may include technologies such as TCP/IP, UDP, WAP, etc.

The backend system 300 may be comprised of one or more servers having one or more processors, operating in conjunction with one or more computer-readable storage media, configured to provide backend services, such as data processing, data storage, data backup, data hosting, among others. The backend system may be configured to split database tables into smaller tables (e.g., monthly tables) to enhance the performance of various searching algorithms and to reduce request response time. Various encryption and security techniques may be utilized to preserve the privacy of the patient information stored on the server.

In some embodiments of the invention, the backend system 300 is a set of distributed computing devices connected through a communications network. An example of such a set of distributed computing devices would be what is typically known as a ‘cloud computing’ implementation. In such a network, a plurality of connected devices operate together to provide services through the use of their shared resources. Different components of the network may have different roles, such as backup roles to help ensure the continual accessibility of the system. Load balancers may also be utilized to control which of the computing devices receives a request and to help ensure that traffic remains consistent.

The support interface 202 provides an interface for the HCT (e.g. clinicians and care providers) to interact with the backend systems through the communication of data. The communication of data may include, among others, the ability to enter data relevant to the care of a particular patient (e.g. inventory and supplies used, notes about their treatment, incidents occurred, limits on permissible inventory). Any data provided by the patient through the patient interface 208 is accessible to the particular HCT caring for a patient through the support interface 202. The HCT is able to use the support interface 202 to schedule patient appointments, communicate directly with patients, update patient information such as prescriptions or orders, or customize patient settings. These patient settings may include the different treatments that a patient will see on their patient interface. The settings can be individualized to also provide specific instructions to the patient that may differ from the instructions that other patients in the same program receive. An example interface may provide various tools that may help facilitate to the development of JSON (Javascript Object Notation) dictionaries by practitioners that may then be used to control what the patient sees in the patient's patient interface. Patient settings may include, but are not limited to: which inventory items to manage, which dialysis modality the patient is currently using, and what steps or stages the patient is expected to go through during a treatment. A change in patient settings may cause a corresponding change in the view and options provided by the patient interface 208 for that particular patient. For example, where one patient is using continuous ambulatory peritoneal dialysis (CAPD) and another patient is using home hemodialysis; the settings may be switched to CAPD for the patient undergoing CAPD and home hemodialysis for the patient undergoing home hemodialysis. The patients may see different information when the patients begin their treatments, and may be required to enter different sets of data. This information, in some embodiments, may be controlled by the JSON dictionaries.

To avoid hospital clinicians/administrators having to spend excessive time customizing the treatments for their patients, default templates may also be provided for the quick introduction of new patients. Templates may be provided to the hospitals for common sets of data. In some embodiments, healthcare facilities may have ability to create customized dictionaries based off of common templates for certain patients (e.g., those that may have unique circumstances).

Further, the support interface 202 may be configured to provide different users with a layout and functionality specific to their needs and responsibilities within the healthcare ecosystem (e.g. a doctor would have a different workflow than a nurse or an administrator).

The support interface 202 may be implemented using a variety of technologies and may be available through more than one platform. For example, the internet may be utilized to provide a web portal for the support interface 202. In some embodiments, the support interface 202 may be accessible through mobile devices, which may be advantageous in permitting the HCT to make quick changes to patient settings without needing to access a computer with an internet connection. The portals provided for mobile devices can likewise be customized for the role of the user in the healthcare ecosystem.

The one or more electronic medical records (EMRs) databases 204 may interact with the backend system 300 in the sharing of data relevant to patients. The interaction may be at varying levels of integration; for example, in some embodiments of the invention, the interaction may involve simply pulling data from external EMRs, and in other embodiments of the invention, the interaction may extend to both pushing and pulling data. The one or more EMR databases 204 are optional as the backend system 300 may also be able to operate as its own standalone medical record.

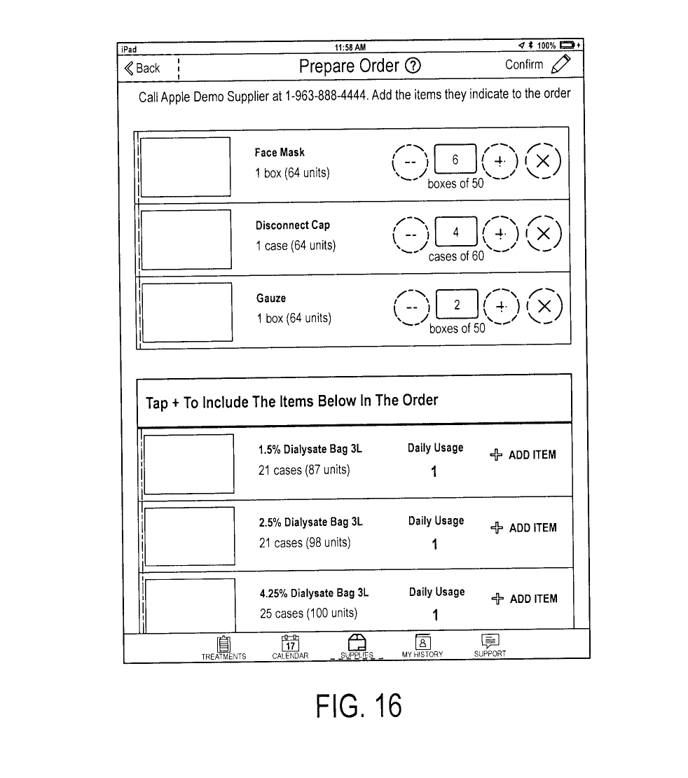

The one or more industry partners/supplier databases 206 may interact with the backend system 300 in communicating information relevant to supplies and inventories of products relevant to the treatment of one or more patients. For example, a patient, through the patient interface 208, may be able to submit an order for a bandage through the backend system 300. In this example, the backend system 300 would then send a supply order to the one or more industry partners/supplier databases 206, and the one or more industry partners/supplier databases 206 may submit one or more invoices in return. FIG. 16 is a sample screenshot of an interface for preparing an order for shipment to the patient, according to some embodiments. The preparation of an order may be split into two sections, one section for preparing an order (including items to be placed in a potential order), and a second section for selecting items to be placed in the order. The items shown in the second section may be prospective items, and in some embodiments, various algorithms may be utilized to determine the types of products relevant to a patient based on information received about the patient, such as the particular chronic condition, modality, etc.

The patient interface 208 may provide a number of different features to patients, according to some embodiments of the invention. Patients may be able to input data into the patient interface 208 before, during and following each treatment; this data may be stored locally and uploaded to the backend system 300. The data is then made accessible to clinicians and other members of the patient's care team through the support interface 202. The backend system 300 may be configured such that data to be stored can be adjusted to match the needs of the patient's care team. For example, different patients may require the use of different dialysate solutions and volumes for their treatments. The JSON dictionaries used in the server can indicate which of the different dialysate bags should be shown to the user for them to choose from.

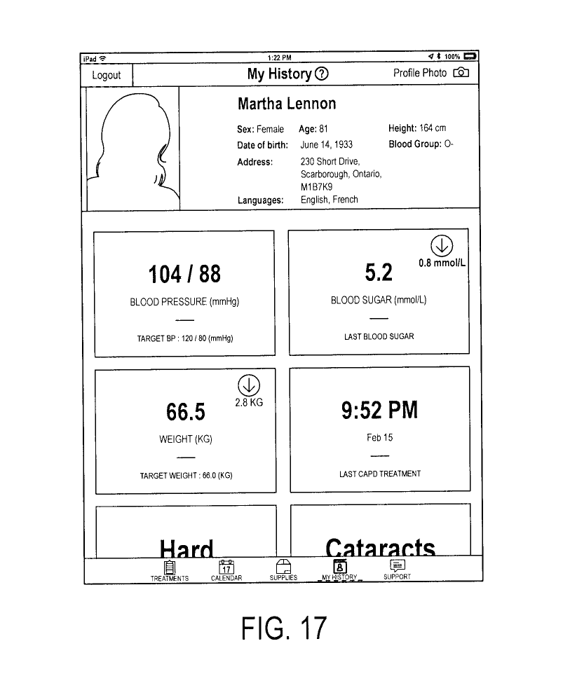

The patient interface 208 may monitor a patient throughout the whole course of his/her treatment (beginning treatment, during treatment, ending treatment, aborting treatment), and provide notifications, reminders, information, and process management, among other features. FIG. 17 is a sample screenshot for an interface providing a dashboard view of information relating to the care of a patient, according to some embodiments. Where a patient encounters an unexpected situation, such as accidentally dropping an object that should remain sterile during the treatment or forgetting how to do a certain procedure, the patient interface 208 may be able to provide guidance. Because each patient may be prescribed different steps and instructions based on their own unique circumstances, this information and process management can be controlled by one or more practitioners through the support interface that facilitates the adjustment the JSON dictionaries that may propagate to the patient interface, changing the instructions and processes that the patient is required to perform.

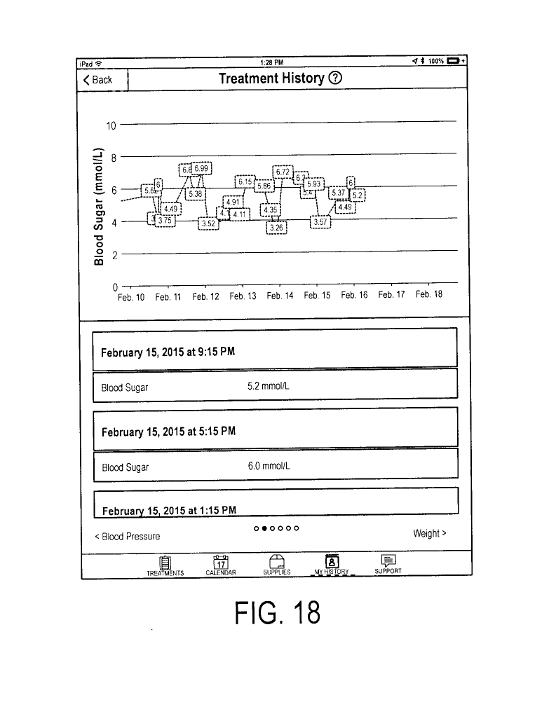

In some embodiments of the invention, the patient interface 208 provides the patient with a graphical representation of information relevant to the course of treatment, such as the progress of their treatment, historical treatment information, amount of inventory remaining, etc. In further embodiments of the invention, the graphical representations are interactive. Notifications and alerts may be generated by the patient interface 208 based on patient status. FIG. 18 is a sample screenshot of a patient's blood pressure level charted against time, according to some embodiments.

Data provided to the patient interface 208 may include clinical data, inventory data, treatment data, diet data, exercise data, and any other data relevant to the health of a patient. In some embodiments of the invention, this data may be provided by the patient's EMR. Depending on the specific procedure type, different data can be collected from the patient in stages; different instructional information can also be presented to the patient in stages. These stages may help prevent the user from skipping steps and potentially enforce proper technique; they also may help improve data collection from the user. The patient may be reminded if any information is missing or filled in incorrectly.

The patient interface 208 may allow patients to enter their physiological measurements (e.g. weight, blood pressure, blood sugar, etc.) at any time during the day. This information may be entered manually or retrieved automatically by the patient interface 208 from various compatible medical devices. The patient interface 208 may retrieve treatment information and troubleshooting records directly from compatible health care machines. The patient interface 208 may also provide standardized, multiple choice options for a field such as for exit site observations (e.g. swelling, redness, pain) that allow patients to more efficiently describe their situations using clinical descriptors and keywords. These input schemes, for example, may help prevent the entry of invalid or erratic information. The ease of entry may help promote patient compliance and the standardized selections may help reduce and/or prevent the entry of irrelevant data, allowing the data to be streamlined and easier to review for practitioners.

In some embodiments of the invention, machine learning algorithms may be applied to patient inputs: over time, this can be used to suggest or pre-fill expected inputs from the user to save time; this can also be used to ensure the accuracy of inputs and any unreasonable entries may trigger alerts to the HCT. These machine learning algorithms may be flexible, and can be configured receive input or instruction from practitioners, for example, providing indications of priority, precedence, weighting, etc., for alerts that the practitioner may feel require more immediate attention.

When a new patient is introduced to patient interface 208, a clinician or other staff member may create a user for them using the support interface 202. The new patient may be requested to provide information, this includes their full name, address, phone number if applicable, password, gender, age, target weight, target blood pressure, and treatment type (for example, dialysis modality, if the patient is on dialysis) for the patient. Depending on the information provided, the patient interface 208 may be prepopulated depending on the specific needs of the patient and depending on the settings set by the HCT, future treatments may be scheduled, and inventory items ordered.

In some embodiments, the patient interface 208 is configured to operate on a network, such as the internet or an intranet. The patient interface 208 may also be configured for use on a mobile device, potentially taking advantage of various technologies such as touch screens to facilitate data entry with multiple-choice selection tools and to provide connections to peripheral devices. Mobile devices may include various devices, including laptops, tablet computers, handheld wireless devices, etc. An advantage provided by the mobile devices is the ability to securely store inputs from a patient This enables the patient to use the interface without access to a network. The patient's data may then be synchronized with the backend server when the mobile device detects an internet connection.

The patient interface 208 may be configured to provide education materials and guides for the patient to follow through their care, using various learning media. In some embodiments of the invention, specific portions of learning media are provided to the patient, the specific portions selected through their relevance on either the patient's condition or the status of their treatment. A practitioner may also be able to use the support interface to provide their own education materials to any number of their patients. Patients may then be able to receive these materials through the patient interface the next time their device synchronizes with the backend server.

The patient interface 208 may also be configured to interface with dialysis machines 216 and other medical devices (e.g. weight scales 212, blood pressure monitors 214, glucometers, etc.) via a multitude of current and legacy connection protocols including Bluetooth, Bluetooth low energy, USB, serial, infrared, NFC, WiFi, ANT+, etc, or one or more appropriate application programming interfaces (APIs).

Further, the patient interface 208 may also, through the backend system 300, be configured to exchange information regarding supplies including inventory levels, placed orders, usage rates, invoices, etc. to the industry suppliers to assist in the provisioning of supplies relevant for their care. This information, for example, may be tracked in a patient profile and accessed for various reasons, such as alert generation, rule definition, input into a neural network, etc.

The patient interface 208 may also be configured to provide tools for patients to interact with one another, for example, using the consumption of the supplies as a basis to match the patient to other patients at a similar stage or have a similar health condition to share insights, or to suggest information relevant to solve a particular problem. In this exemplar embodiment, a patient may be able to use the platform to generate data to be shared with others based upon their personal health data, or more granular updates on their current status based on the state of their inventory, etc.

In some embodiments of the invention, the patient interface 208 presents patients, who are waiting for a treatment procedure to complete, with access to relevant information including procedure instructions, lifestyle advice, advertisements (new products, vacation offers, etc.), e-magazines, news, research, community network, etc., which can take various forms of media including text, audio, images, video, animation, games, interactive interfaces, etc.

The actual interface elements available through the patient interface 208 may be controlled and customized by the HCT to whom a patient is assigned. These controls are available through the support interface 202, and may be downloaded directly from the backend system 300 without having the patient travel to the hospital, as long as the patient's device has access to the network (e.g. the internet). As a result, the interface elements provided to each patient through the patient interface 208 may be tailored towards the circumstances of the patient.

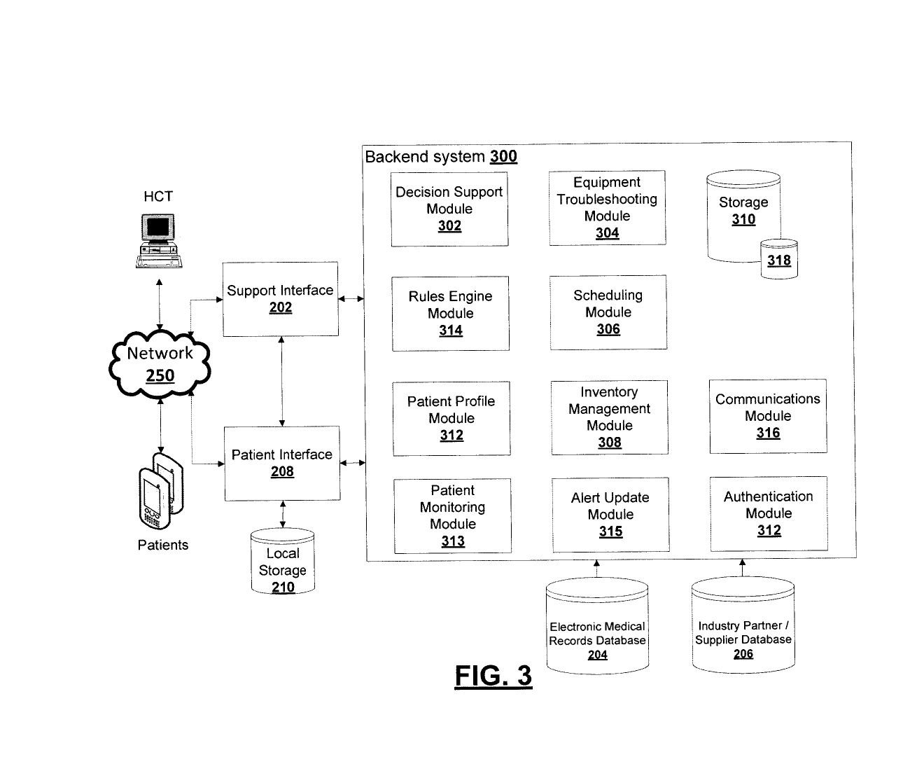

Referring to FIG. 3, a block diagram illustrating a backend system 300 is shown, according to some embodiments of the invention.

The backend system 300 may be comprised of a decision support module 302, an equipment troubleshooting module 304, a scheduling module 306, an inventory management module 308, one or more storage elements 310, an authentication module 312, a patient profile module 313, a rules engine module 314, a patient monitoring module 315, a communications module 316, an alert update module 317, and a local storage element 210.

The local storage element 210 stores information related to the patient interface 208. Information such as patient inputs, educational materials, clinical information, is saved locally so that a patient is able to access the data without a network connection present. Furthermore, data saved on the local storage element 210 may be uploaded at a later time when a network connection is present to the backend system 300.

The decision support module 302 may be configured to provide data analytics to both patients and the HCT, through the patient interface 208 and the support interface 202, respectively. The decision support module 302 may be configured to provide historical data, provide views on old treatment logs, conduct statistical analysis, data transformations, respond to queries, develop reports (e.g. usage, financial costs related to care), provide detailed support with respect to a particular condition or status, provide calculators based on patient status, develop visualizations (e.g. graphs of usage over time), track patient behaviours (e.g. location using wifi, touch gestures, time spent on different screens and manuals), provide analysis on patient behaviours (e.g. pinpointing which manuals are more popular and successful for given situations, or how the patient interface may be improved) and provide incident/threshold alerts, among others.

In some embodiments, the decision support module 302 is also configured to provide suggestions/decision support to the user based on their inputs and settings. For example, a tag selection calculator′ for peritoneal dialysis patients may suggest which concentration of dialysis solution (or bag) to select for their upcoming treatment procedure. The suggestion may be formulated based on multiple factors including weight, blood pressure, fluid status, whether it is a day or overnight treatment, etc. The patient interface 208 may present this information to the patient in a graphical, easy to interpret manner, emphasizing key inputs/settings and the resulting suggestion.

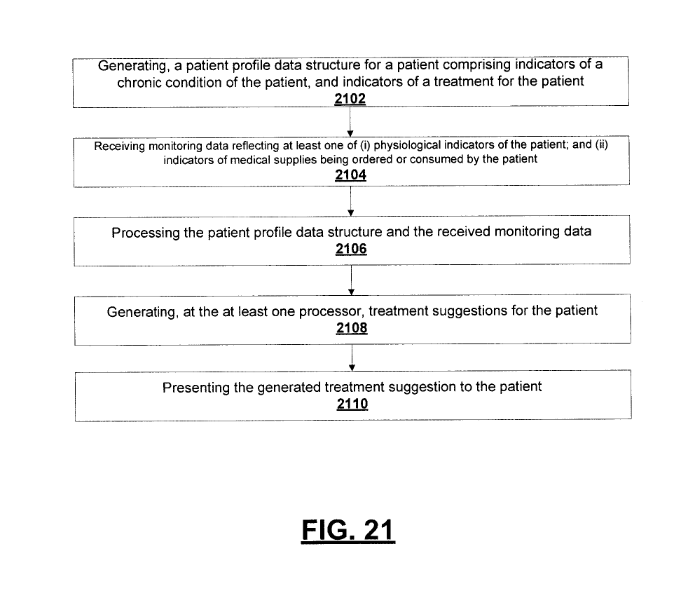

FIG. 21 provides a sample workflow for the generation of treatment suggestions to a patient, according to some embodiments. At step 2102, the system may be configured for generating, at at least one processor, a patient profile data structure for a patient, the patient profile data structure comprising: indicators of a chronic condition of the patient, and indicators of a treatment for the patient. At step 2104, the system may be configured for receiving, at the at least one processor, monitoring data reflecting at least one of (i) physiological indicators of the patient; and (ii) indicators of medical supplies being ordered or consumed by the patient. At step 2106, the system may be configured for processing, at the at least one processor, the patient profile data structure and the received monitoring data. At step 2108, upon said processing, the system may be configured for generating, at the at least one processor, treatment suggestions for the patient. At step 2110, the system may be configured for presenting the generated treatment suggestion to the patient.

The decision support module 302 may also help troubleshoot alerts by displaying possible solutions that have worked in the past, based on previous alert patterns and when during the treatment the alert occurred. These alerts can link directly to patient education materials and guides to help the patient solve the encountered problem.

The equipment troubleshooting module 304 may be configured to connect with compatible health care devices, peripherals and machines, receiving outputs, including machine alerts. These machine alerts may be input into learning algorithms that take into account when during a treatment the alert occurred, previous alert patterns, among others, and then display to the user solutions that may have been effective in the past to resolve this specific alert during this specific time and under the same conditions.

As an example, an algorithm may provide the following workflow: First, the equipment troubleshooting module 304 receives an alert from the machine. Second, the equipment troubleshooting module 304 identifies the source or cause of the alert. Third, the equipment troubleshooting module 304 checks the stage/step that the patient is currently on and the surrounding conditions. Fourth, the equipment troubleshooting module 304 searches the database for matches of this alert occurring during this step. Fifth, the equipment troubleshooting module 304 retrieves any solutions that may be successful at resolving the patient's situation or have worked for other patients in similar situations. Sixth, if solutions are found, the equipment troubleshooting module 304 refers these solutions to the patient; otherwise the equipment troubleshooting module 304 informs the patient to contact their clinician for assistance. Seventh, when the patient resolves the problem, the equipment troubleshooting module 304 requests feedback through patient interface 208 as to how the problem was solved. The solution is stored by the backend system 300 for reference for future patients.

In some embodiments of the invention, the algorithm may refine the approach taken to troubleshoot issues through obtaining both direct and indirect feedback from time spent looking at different manuals, location of the patient (e.g. using wifi, GPS or other location tracking technologies), touch gestures used by the patient, time spent on specific pages, last manual accessed before the treatment restarted (i.e. alert resolved), and direct acknowledgement of successful and unsuccessful attempts. Other valuable information gathered from the equipment by the algorithm would include which alerts were solved or not solved, and how much time was taken to solve different alerts. This information may be stored by the backend system 300 as the information may be beneficial towards patient retraining, quality improvements to improve training for new patients, and for product development/improvement by suppliers.

The scheduling module 306 may be configured to provide either the HCT, through the support interface 202, or the patient, through the patient interface 208, the ability to perform scheduling activities, such as setting up appointments, cancelling appointments, appending notes/messages to appointments, etc. Appointments may be in person or, in some embodiments of the invention, over telecommunications protocols. The scheduling module 306 may be further configured to provide automated reminders with respect to appointments, or, reminders with respect to particular actions related to the care of a patient. For example, a patient may be reminded on a periodic basis to start the course of his/her treatment, or, on, when the event arises, to purchase more bandages because the backend system 300 has detected that the patient will be low on bandages following this course of treatment. The scheduling module 306 may also be used for reminders that a particular shipment of inventory is due to arrive.

Examples of medical related events including but not limited to: treatment procedures, equipment maintenance/tests, nurse home visits, technologist home visits, personal support worker home visits, clinic & doctor appointments, supplies delivery, supplies reordering, scheduled video calls with a clinician. The scheduling module 306 may also provide alerts to help notify/remind the user ahead of time of upcoming events.

The scheduling module 306 may also be configured to help a patient prepare and plan for any upcoming trips/vacations (multiple nights spent away from home) that they want to take. For example, with peritoneal dialysis, the majority of planning will be around having an adequate supply of dialysate bags in the non-home location for the patient to perform their regular frequency of dialysis treatments.

The inventory management module 308 may be configured to provide up-to-date inventory tracking; analytics based upon the historical usage, wastage and consumption patterns related to inventory; and inventory ordering capabilities. The inventory information may be prepopulated based on a particular method of treatment for a patient, The inventory information may be monitored through every step of treatment, stored in the backend system 300 and shared with patients through the patient interface 208, the HCT through the support interface 202, and the one or more industry partners and suppliers. Features to be managed include supply levels, usage rates, maintenance and service procedures performed, supply reordering, supply wastage, expiry dates, etc. Items may be presented to the user with a picture of the item, item name, supply level, instructions for the proper use and care of the item, etc.

The inventory management module 308 is linked to the rules engine module 314 in applying and developing rules related to inventory. For example, rules may be applied to historical data to interpolate data for a particular user to determine average usage rates for particular items, or these rules may be applied to determine that a patient is using a particular item inconsistently when compared across the broader population of all patients with similar circumstances.

In addition to selecting items to order, the inventory management module 308 may implement an algorithm that recommends other items that are low in stock or close to low and may need reordering soon; potentially increasing order efficiency and potentially reducing delivery expenses. The algorithm may consider supply usage rate, current inventory levels, next scheduled order, the predicted remaining amount at the time of the next scheduled order and the predicted maximum length of time from order date to delivery date to determine whether a user will run out of an item before the next scheduled delivery. The algorithm may also deter orders that contain items that would expire based on the quantity ordered, item expiry date, and average usage rates. The algorithm may also deter against excessive orders to prevent patients from stockpiling supplies unnecessarily.

In some embodiments, the inventory management module 308 may automatically order items based on the applied rule set.

The rate at which certain items are consumed in relation to other items is determined by the inventory management module 308. For example, if facemasks or alcohol swabs are used at a lower rate than expected and/or at a lower rate than other supplies, this may indicate that the patient is not following proper sterile procedure during their treatments. This inventory management module 308 may be configured to identify these cases and notify/alert clinicians ahead of time that retraining may be necessary for this patient. The inventory management module 308 may also be configured to provide a retrospective analysis after a patient acquired an illness (e.g. peritonitis infection); potentially aiding clinicians in determining a likely cause of illness by examining patient inventory usage rates. The information produced by the algorithm may also be used for continuous quality improvement purposes to improve the training for new patients. This algorithm, in some embodiments, includes the addition of audit tables into the database. The audit tables may be designed such that whenever an item is changed or created, an audit record is created that stores some or all the information of the new changed or created item, as well as the cause of the change or creation, and records the user that effected the change and creation.

Depending on which stage/step of starting the treatment procedure a patient is in, or how far into the treatment the patient aborts at, the inventory management module 308 may determine which supplies have been consumed already and which supplies haven't been used yet and can be recovered and reused for a restarted or cancelled treatment. This information may be presented to the patient to reduce wasted supplies, and may also be forwarded to suppliers and hospitals as information on patient error rates and waste. The supplies used up in the aborted treatment can be automatically deducted from the patient's inventory, after the patient provides confirmation.

As a patient completes each stage/step of his/her treatment, a list of the supplies that would have been used during that step is logged by inventory management module 308. If a patient aborts the treatment, all the supplies that have been logged in the list for that particular treatment are considered potentially used. Some supplies may be reusable but those that are not are flagged to be thrown away by the patient and are deducted from their inventory. The patient may indicate that an item was not actually used (perhaps this may have been the reason for aborting the treatment in the first place). If so, the item is not deducted from the patient's inventory.

As a patient obtains and uses inventory, the changes to the inventory are logged into the local database and uploaded to one or more storage elements 310. These logs indicate who effected the changes and how they were caused. By interpolating all the data for a particular user, an average usage rate can be found for a particular item. The average usage rate may then be extrapolated to estimate future needs of the patient, such as how much that patient should stockpile of a particular supply item, supply consumption related to demographics and seasonal changes, or how often a patient's machine will require maintenance or replacement. The usage rate of each patient may also be compared with the average rates of other patients to identify any inconsistencies for a particular patient. This tracking system may be particularly useful for tracking multi-use supply items, such as hand sanitizer. For example, the system may be configured to determine what the average rate of use of hand sanitizer would be, which could be used to estimate the length of time a patient could use one bottle of hand sanitizer before it is empty and needs to be replaced.

The one or more storage elements 310 may also be configured for the storage of various patient information, such as patient profiles, records, inventory usage logs, care logs, electronic medical records, device records, patient history, alerts, alert rules, etc. In some embodiments,

The data held in the backend system 300 from patients including the procedure rate, time of day, duration, user patterns, time of year, number of alerts, cancellation rate, etc. may be used to predict the usage/consumption of supplies during the treatment. The predicted supplies used for the treatment may be automatically deducted from a patient's inventory, after patient confirmation/adjustment. This information may also be used to estimate the wear on multiple-use supplies/equipment and predict when these components will need replacing/maintenance, and notify the appropriate users (patients, care team, administrators). This information can also be used by users to predict/plan for future supply usage rates at an administrative level.

Using general patient averages, actual usage rates specific to each patient, and treatment information entered by the patient, the inventory management module 308 is able to calculate the typical supplies used in a given treatment. Thus, when a treatment is performed for a patient, supplies are automatically deducted from the patient's standing inventory. The patient may also make manual corrections if their supply use was different from the calculated values for the logged treatment, or if they discover unusable equipment that must be disposed of such as a leaking dialysate bag or contaminated equipment.

The inventory management module 308 may be configured to operate with the patient interface 208 to request periodic inventory input in the form of request triggers, requested by the HCT, as well as automatic triggers. An automatic trigger occurs when a patient wants to place an order for more supplies. The patient interface 208 will ask a patient to do a manual count before each order to verify which items they have enough of to last to the next order delivery date. The reasoning for the timing of this automatic trigger is twofold: patients should be double-checking before an order that they are actually low in that item to avoid unnecessary orders; the period before ordering an item is when inventory levels should be at their lowest and thus should be the easiest time to do a manual count.

The one or more storage elements 310 may be configured to store and to host data related to the backend system 300, including, but not limited to, patient data, interface settings, pre-populated templates for specific courses of treatment, benchmarking data for inventory usage, benchmarking data for the analysis of particular courses of treatment, educational materials, information on patient behaviours (e.g. location information, time spent doing various tasks or looking at various screens, manuals and information accessed, touch gestures used) information on how to solve issues, information related to attempts at solving issues, past communications between patients and the HCT, invoices for supplies, supply orders, scheduled appointments, notifications, etc. The storage elements may be stored in various formats, such as, but not limited to, relational databases, flat files, enterprise data warehouses, etc. Further, the one or more storage elements 310 may also contain relationships between various data points.

In some embodiments of the invention, the one or more storage elements 310 is a data warehouse that is configured to further support reporting and data analysis by providing the ability to extract, transform and load data, pre-fetch data and utilize data caches as may be understood by one skilled in the relevant arts.

In some embodiments of the invention, the one or more storage elements 310 is designed to provide data backup and data recovery through the use of technologies such as redundant arrays of storage, data striping, etc.

The authentication module 312 may be configured to receive authentication information from users prior to use, at the support interface 202 or the patient interface 208. At the patient interface 208, proper authentication information may be required to access the patient interface 208. At the support interface 202, the authentication information may be utilized to determine which user is using the support interface 202 and that information may be applied in setting up an interface that is designed for the particular needs of that user (e.g. a doctor as compared to a nurse or an administrator). The authentication, for example, may be related to information stored in a patient profile, such as date of birth, secret questions, passwords, etc.

The patient profile module 313 may be configured to generate a patient profile data structure for a patient, the patient profile data structure comprising indicators of characteristics of the patient. Indicators of characteristics, for example, may include various elements of information retrieved from an electronic medical records database 204, historical interactions with the patient interface 208, self-reported information provided through the patient interface 208, recorded information from the HCT provided through the support interface 202, etc. In some embodiments, the patient profile also tracks the patient's historical, current, and predicted supply usage information. For example, the patient profile may also include indicators of medical supplies being ordered and/or consumed by the patient, such as gauze, bandages, needles, dialysate, medical bags, etc. The patient profile may also be associated with information related to the care and/or services provided to the patient, such as records and/or operation logs from dialysis machines, analytical records of biological fluids being provided to and/or removed from a patient, etc.

The rules engine module 314 may be configured to apply various rules that enable remote monitoring of inventory, and the analysis of inventory information to determine whether procedures are being followed properly. For example, upon application of the rules to analyze the data at the backend system 300, it may be determined that a patient (a) has not been following the dialysis ratios; and (b) not using face masks properly. The early identification of these factors may have significant benefits to the health care outcomes for a patient, potentially avoiding negative consequences, such as infections. Further, such an analysis may also potentially enable improvements in management of inventory/delivery, both at patient level and at a supplier level.

For example, rules may having conditions and/or parameters related to/such as: blood pressure, pulse rate, blood sugar, weight (change), weight (range), cloudy effluent, very cloudy effluent, pink effluent, red effluent, fibrin present, exit site redness, tender exit site, painful exit site, exit site swelling, exit site discharge, negative total ultrafiltration, low total ultrafiltration, drain bag weight, comment or problem logged, treatment missed, bloody stool logged, hard stool logged, no stool logged, 4.25% bag selection, 0.5% bag selection, critically low inventory, treatment waste, ordering error—excessive order, ordering error—wrong items, ordering error—missed order, delivery failure, delivery error, new staff message, new patient message, staff video chat request, patient video chat request, equipment alert, calendar event reminder, dwell time, etc.

In some embodiments of the invention, the rules engine module 314 may apply machine learning algorithms to predict the likelihood of certain outcomes based on identified precursors and patient factors.

The patient monitoring module 315 may be configured to receive monitoring data reflecting at least one of (i) physiological indicators of the patient; and (ii) indicators of medical supplies being ordered or consumed by the patient; and generate an alert relating to patient healthcare by applying at least one of the plurality of alert generation definitions to the received monitoring data. For example, an alert may be generated through communications module 316.

The patient monitoring module 315, in some embodiments, continuously tracks the patient and causes updates to the patient profile when new and/or updated information is received. The patient monitoring module 315 may also monitor current inventory levels and also various characteristics of supply logistics to the patient. For example, if patient's profile indicates that the patient receives shipments of supplies only once a month (e.g., the patient lives in a remote location), the patient monitoring module 315 may then be configured to provide alerts when inventory levels are at risk of falling into dangerously low levels during periods where the patient will not be ordinarily receiving shipments. The communications module 316 may be configured to enable communications between the HCT and patients, between HCTs and HCTs, or between patients and other patients; these communications may be scheduled or unscheduled, and may include the sending of messages, establishing discussion forums, voice communications, video communications, among others.

The alert update module 317 may be configured to receive population data reflective of healthcare outcomes for a patient population. For example, one or more alerts may be developed based on the application of one or more business rules. These alerts may contain various conditions and indications of when an alert should be triggered, and what, if any, events should be triggered as the result of an alert. The relationships between healthcare inputs and outcomes can be very complex and non-linear, and accordingly, the relationships themselves are often poorly understood. For example, the definition of rules designed to trigger alerts to prevent deleterious healthcare outcomes (e.g., to determine what conditions lead to a patient's effluent becoming cloudy so that an alert can be promptly triggered) may be difficult. Accordingly, in some embodiments, the update module 317 may be configured to utilize one or more analytical techniques to update alert rules based on information provided for other patients, such as for an overall patient population and/or a sectional patient population (e.g., patients having one or more similar characteristics).

The update module 317 may be configured for monitoring data for the patient population and generating at least one further alert generation definition upon processing the population data; and updating the storage 310 with the at least one further alert generation definition.

The new alert generation definition(s) may be used by patient monitoring module 315 to generate alerts going forward. For example, patient monitoring module 315 may receive further monitoring data and the new alert generation definition(s) may be applied to the further monitoring data.

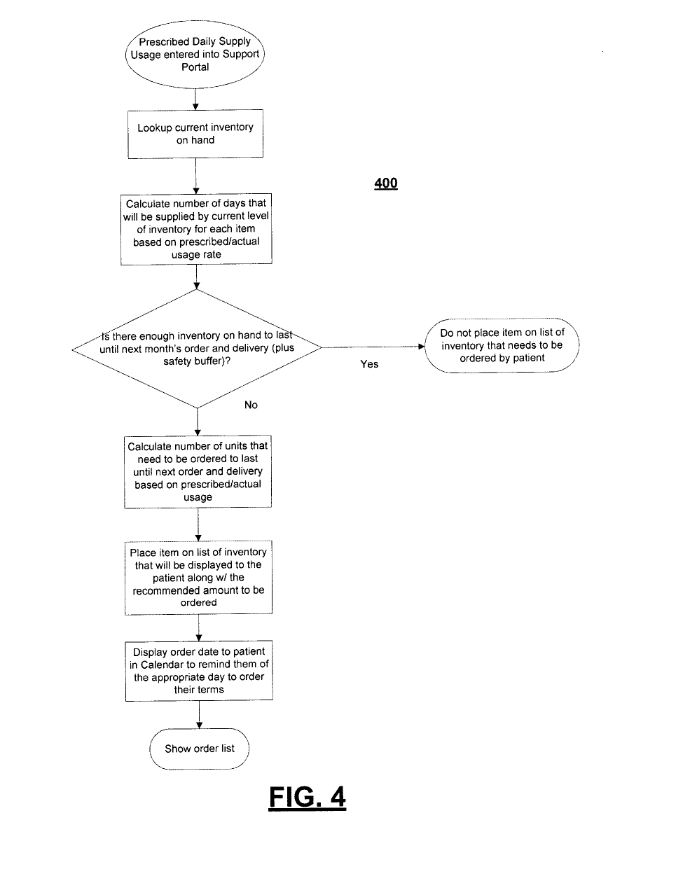

Referring to FIG. 4, an inventory ordering workflow 400 is provided, according to some embodiments of the invention.

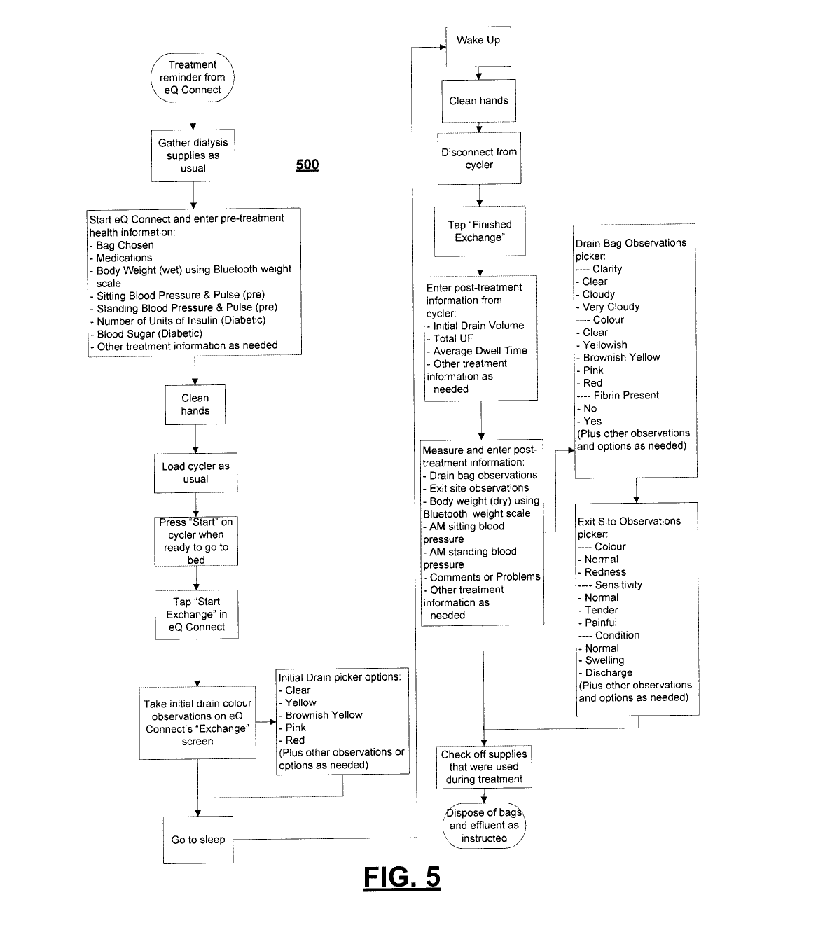

Referring to FIG. 5, an example patient treatment workflow 500 is provided, according to some embodiments of the invention. Workflow 500 is provided for use with Automated Peritoneal Dialysis (APD) treatment, and is provided from a patient's perspective.

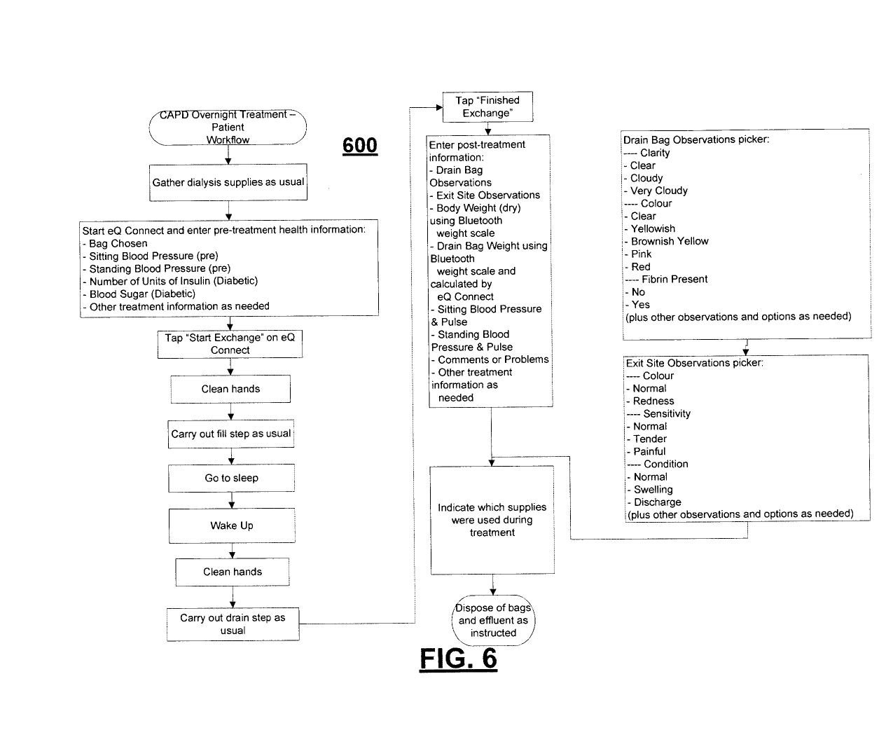

Referring to FIG. 6, an example patient treatment workflow 600 is provided, according to some embodiments of the invention. Workflow 600 is provided for use with CAPD overnight treatment, and is provided from a patient's perspective.

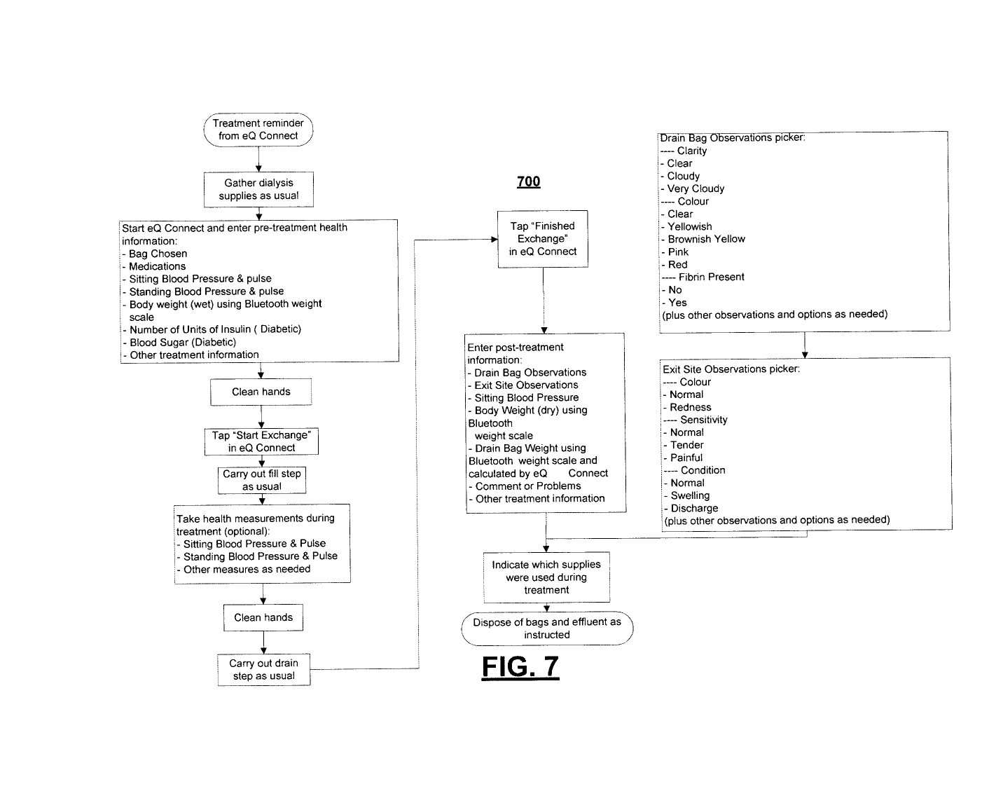

Referring to FIG. 7, an example patient treatment workflow 700 is provided, according to some embodiments of the invention. Workflow 700 is provided for use with CAPD treatment, and is provided from a patient's perspective.

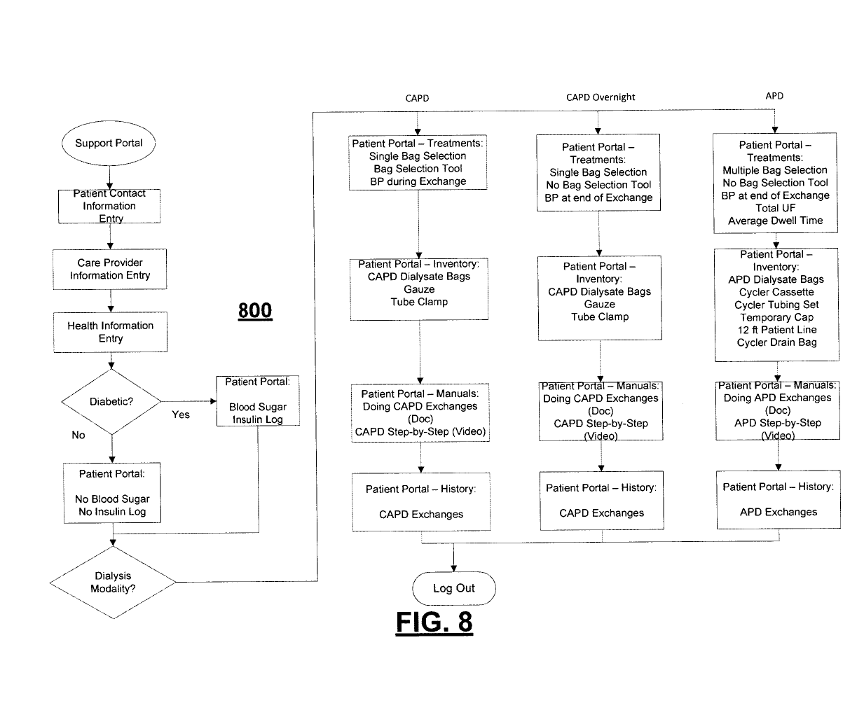

Referring to FIG. 8, an example workflow 800 is provided for using the support portal, according to some embodiments of the invention. Workflow 800 indicates inputs made into the support interface 202 and the resultant effects on patient interface 208.

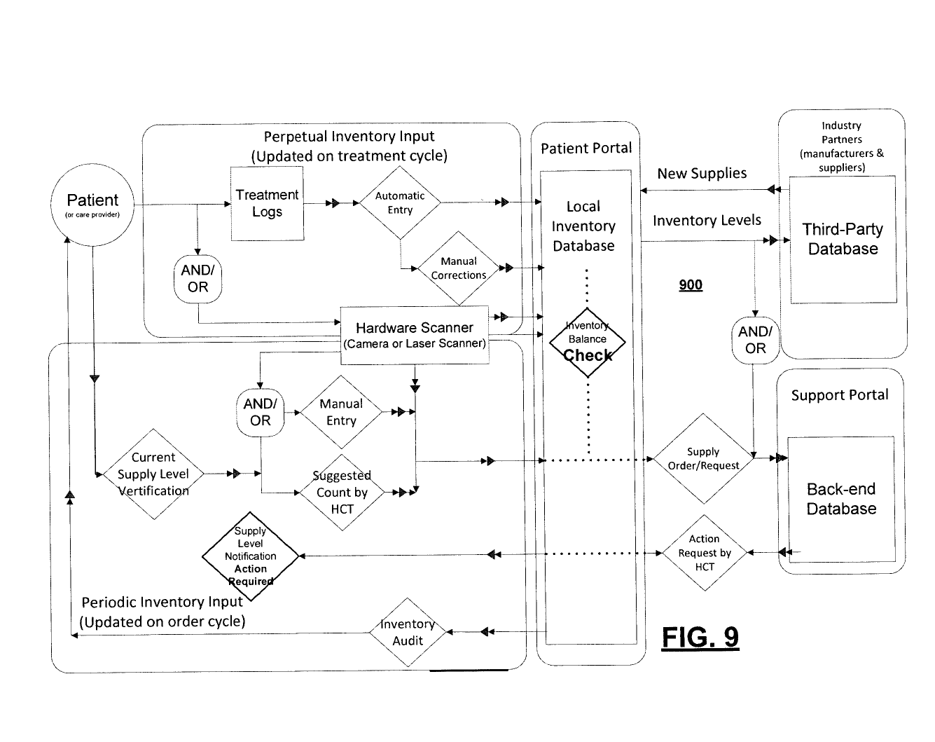

Referring to FIG. 9, an example inventory workflow is provided, according to some embodiments of the invention. FIG. 9 provides for a sample inventory process providing the interactions between the databases and modules.

Referring to FIG. 18, an example alert generation and presentation workflow is provided according to some embodiments of the invention.

### Alert Rule Template Generation Examples

A healthcare environment may be complex, dynamic system including a diverse set of inputs and outputs, as well as myriad interconnections that change over time.

Accordingly, a skilled reader may appreciate that relationships between various inputs, outputs, outcomes, etc., may not be readily definable and/or available. For example, while it may be rudimentary to determine that a patient has lost weight, it may be more challenging to identify relationships that identify the various causes and/or rationales for why the patient has lost weight. There may be a large number of physiological, behavioral, and situational factors involved, some of which may be controllable by healthcare practitioners, and some of which may be chaotic and/or random in nature.

There may be significant value in determining these relationships, however, as the identification of a relationship may aid in promoting various health outcomes (e.g., reduced morbidity among patients). These relationships may be used, for example, for research, for the generation of tailored rules, the identification of risk factors, root cause analysis, etc., and in some cases, active steps may be taken to rectify various deficiencies or to influence various outcomes.

Over a large enough data set, the relationships between various variables may be identified through an iterative refinement and/or adaptation of one or more base assumptions.

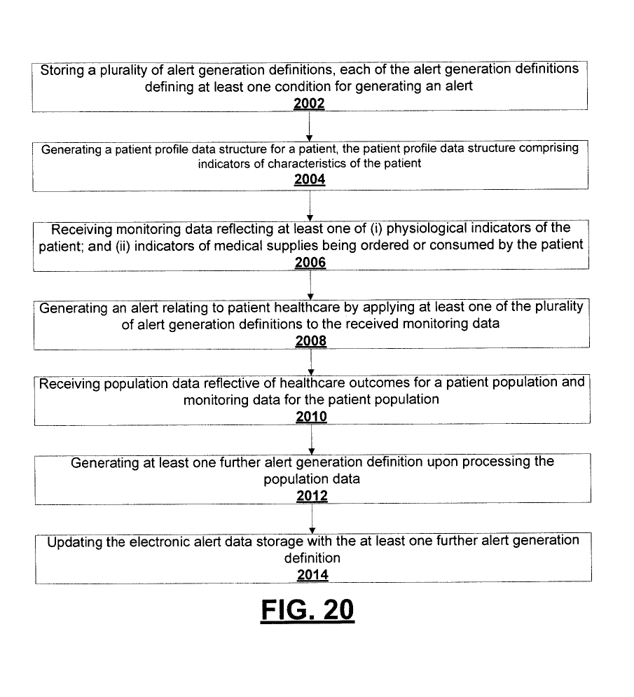

FIG. 20 is a sample workflow illustrating steps that may be performed in updating an electronic alert data storage with further alert generation definitions based on healthcare outcomes for a patient population, according to some embodiments. The system may be configured for the performance of various steps, including: step 2002, storing a plurality of alert generation definitions in electronic alert data storage, each of the alert generation definitions defining at least one condition for generating an alert; step 2004, generating, at at least one processor, a patient profile data structure for a patient, the patient profile data structure comprising indicators of characteristics of the patient, step 2006, receiving, at the at least one processor, monitoring data reflecting at least one of (i) physiological indicators of the patient; and (ii) indicators of medical supplies being ordered or consumed by the patient; step 2008, generating, at the at least one processor, an alert relating to patient healthcare by applying at least one of the plurality of alert generation definitions to the received monitoring data; step 2010, receiving, at the at least one processor, population data reflective of healthcare outcomes for a patient population and monitoring data for the patient population; step 2012, generating, at the at least one processor, at least one further alert generation definition upon processing the population data; and step 2014, updating, at the at least one processor, the electronic alert data storage with the at least one further alert generation definition.

In some embodiments, one or more methods may be provided in relation to the use of support vector machines (SVMs) to train a neural network over various inputs.

The neural network may be configured for receiving information related to various inputs (e.g., physiological data, machine readings, self-recorded data) and outcomes (e.g., patient death, patient side effects, renal failure, cloudy effluent discharge).

This information may be used in the configuration and/or definition of one or more alerts, where the alerts are defined by a number of conditions related to various aspects of a patient's profile or supply information. The alert may be triggered, for example, when a number of conditions are met. The conditions may have various weights assigned to them, and for example, an alert may be triggered only when the aggregate of weights of satisfied conditions is greater than a particular threshold. These weights may be varied.

The weights allow for rebalancing of the conditions, which may be used to adapt the alert conditions in view of healthcare outcomes that are very complex and difficult to model. As it is often unclear as to the exact cause or mechanism of action for a particular outcome, adaptive rules may allow for the adaptation of rules that may be adapted over time to be more effective at tracking the relationships between data and an outcome. For example, a potential outcome may be the presence of cloudy effluent from dialysis, but it may not always be clear what conditions exactly causes the cloudy effluent, or there may be multiple conditions that need to be present together.

The neural network may be configured to utilize one or more feedback loops to associate outputs with inputs, and to refine its analysis through recursive iterations through various data sets.

For example, the neural network may be provided with an initial prediction, or an initial set of predictions that are related to one or more outcomes associated with the one or more conditions. The initial predictions may be used to map a set of base relationships between inputs and outputs form an initial set of predicted outcomes. The base relationships may include various weightings, which, in some embodiments, may be randomly assigned based on various rules. As actual healthcare outcomes are logged and recorded by the system, the neural network may be configured to compare the actual outputs to predicted outputs. Based on the difference between the predicted outputs and the actual outputs, the neural network may then be configured to change the values of the weights so that the predicted outputs would more closely align to the actual outputs. The assignment of weights to various relationships may be considered a learning rule, and the learning rule may iterate in relation to data captured for interactions with one or more patients.

The training process may be repeated across a large number of patients, until the neural network either reaches a threshold level of accuracy, or a predetermined number of iterations and/or period of time has elapsed.

The relationships and/or condition weights determined by the neural network may be indicative of population level trends, previously unknown relationships, etc.

As an example, the neural network may be trained to determine the relationships related to a detected cloudy effluent flow from a dialysis machine. First, a hypothesis function may be generated, the hypothesis function developing a prediction in relation to cloudy effluent flow. The hypothesis function may, for example, include a number of relationships that may be generated between various elements of information that may be input into the neural network, such as body weight, change in body weight, dietary information, time of day, age, type of dialysis, supply consumption information, supply availability information, etc. These function parameters, for example, may be conditions associated with whether an alert is triggered.

Each of these relationships may be assigned a weight, and the function may use the weighted relationships to generate a predicted outcome. For example, the outcome may be the level of cloudiness in effluent.

Over a period of time, the system may receive and/or record information related to a number of patients, some of whom may have cloudy effluent. For each, or a portion of these patients, the system may iterate the neural network function to predict how cloudy the effluent flow for a patient may be. In some embodiments, a training set is used with the neural network so that the neural network may train by iterating over historically recorded data sets.

The actual cloudiness of effluent flow may be measured, and compared against the predicted cloudiness. Where there is a difference, the weighting of the values between relationships in the function may be modified such that the error between the predicted outcome and the actual outcome is reduced. The adjustment of the weights may be configured, for example, through one or more random processes that may, for example, perturb various weights slightly and re-run the analysis to determine impacts on the observed error.

In some embodiments, the neural network is used in relation to the monitoring of dialysis, and in particular, the monitoring of supply information related to dialysis. Various elements of supply information may be received from the system, including existing inventory levels, historical inventory levels, order information, etc. There may be various outcomes associated with patient supply ordering that may not be readily apparent. For example, an increased usage of a particular type of dialysis supplies (e.g., masks, disinfectant, solution, drain bags, tubing), combined with other physiological indicators (e.g., sudden weight loss, fistulas), may be related to cloudy effluent.

The neural network may be able, over a period of training, determine this relationship, and upon investigation, an issue may be uncovered that may allow the HCT to provide care that may preempt a bacterial infection that may be indicated by the presence of cloudy effluent.

The tracking of medical supply information and use of medical supply information as inputs, in conjunction with physiological indicators into a neural network for training may help in the determination of relationships that would otherwise be difficult to detect.

Output from the neural network may be fed back into the AlertRulesTempates to be instantiated into AlertRules for a given patient. As an example, the neural network may be configured to produce a function which may identify that patients who order more than 3 packages of tape per month will come in over budget. Running this function across data for a population-level (e.g., all or a subset) of patients, it may be determined that it is true in 80% of cases. Accordingly, the AlertRulesTemplate may be developed for instantiation into one or more AlertRules, which when triggered, indicates to the HCT that the equipment usage of the patient should be checked or re-evaluated.

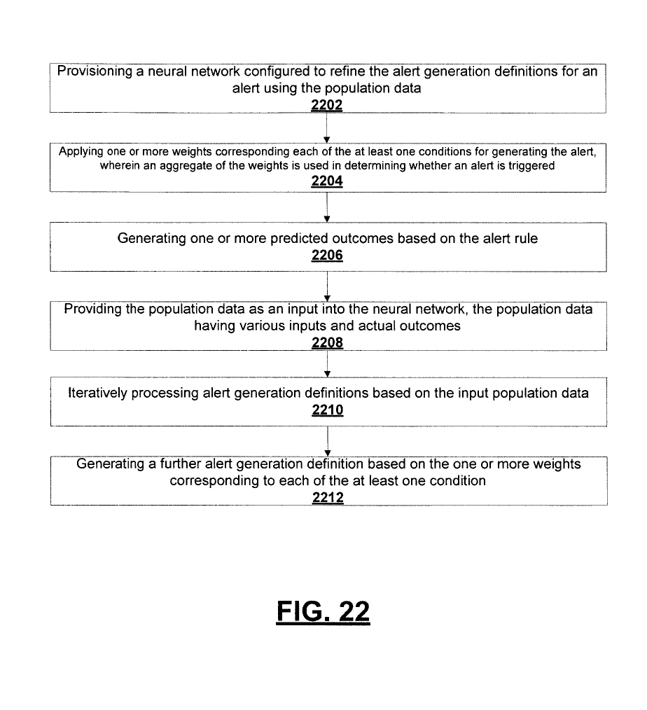

FIG. 22 is an example workflow of the generation of alerts using a neural network, according to some embodiments. In this example, the use of the neural network includes the step 2202 of provisioning a neural network configured to refine the alert generation definitions for an alert using the population data; step 2204 of applying one or more weights corresponding each of the at least one conditions for generating the alert, wherein an aggregate of the weights is used in determining whether an alert is triggered; step 2206 of generating one or more predicted outcomes based on the alert rule; step 2208 of providing the population data as an input into the neural network, the population data having various inputs and actual outcomes; step 2210 of causing the neural network to iteratively process alert generation definitions based on the input population data, wherein the one or more weights corresponding to each of the at least one condition are varied, and on each iteration: using population data related to an individual in a population, determine whether each of the at least one conditions for generating the alert corresponds to a related actual outcome, if so, increase the weight corresponding to that condition, and if not, decrease the weight corresponding to that condition; and step 2212 of generating a further alert generation definition based on the one or more weights corresponding to each of the at least one condition following the use of the neural network to process iterations of alert generation definitions. In some embodiments, the outcome of step 2212 is provided as an input into the neural network so that the neural network may further refine its output functions.

### General

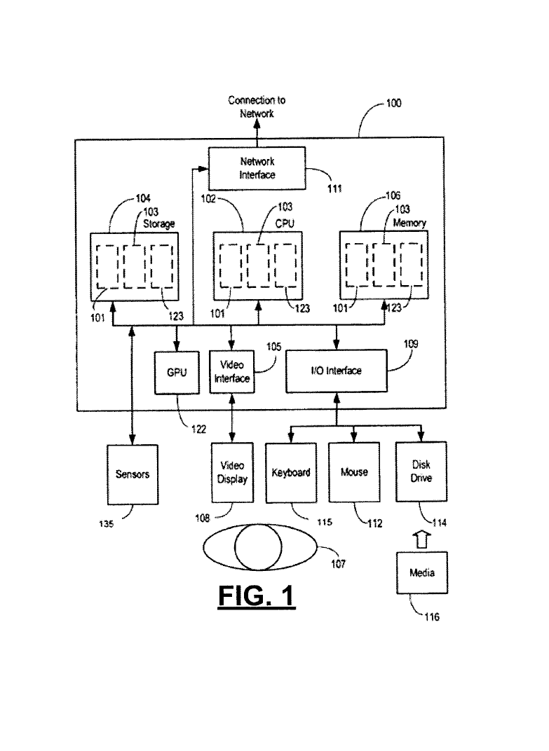

The present system and method may be practiced in various embodiments. A suitably configured computer device, and associated communications networks, devices, software and firmware may provide a platform for enabling one or more embodiments as described above. By way of example, FIG. 1 shows a computer device 100 that may include a central processing unit (“CPU”) 102 connected to a storage unit 104 and to a random access memory 106. The CPU 102 may process an operating system 101, application program 103, and data 123. The operating system 101, application program 103, and data 123 may be stored in storage unit 104 and loaded into memory 106, as may be required. Computer device 100 may further include a graphics processing unit (GPU) 122 which is operatively connected to CPU 102 and to memory 106 to offload intensive image processing calculations from CPU 102 and run these calculations in parallel with CPU 102. An operator 107 may interact with the computer device 100 using a video display 108 connected by a video interface 105, and various input/output devices such as a keyboard 115, mouse 112, and disk drive or solid state drive 114 connected by an I/O interface 109. The mouse 112 may be configured to control movement of a cursor in the video display 108, and to operate various graphical user interface (GUI) controls appearing in the video display 108 with a mouse button. The disk drive or solid state drive 114 may be configured to accept computer readable media 116. The computer device 100 may form part of a network via a network interface 111, allowing the computer device 100 to communicate with other suitably configured data processing systems (not shown). One or more different types of sensors 135 may be used to receive input from various sources.

The present system and method may be practiced various computer devices including a desktop computer, laptop computer, tablet computer or wireless handheld. The present system and method may also be implemented as a computer-readable/useable medium that includes computer program code to enable one or more computer devices to implement each of the various process steps in a method in accordance with the present invention. In case of more than computer devices performing the entire operation, the computer devices are networked to distribute the various steps of the operation. It is understood that the terms computer-readable medium or computer useable medium comprises one or more of any type of physical embodiment of the program code. In particular, the computer-readable/useable medium can comprise program code embodied on one or more portable storage articles of manufacture (e.g. an optical disc, a magnetic disk, a tape, etc.), on one or more data storage portioned of a computing device, such as memory associated with a computer and/or a storage system.

The mobile application of the present invention may be implemented as a web service, where the mobile device includes a link for accessing the web service, rather than a native application.

The functionality described may be implemented to any mobile platform, including the iOS™ platform, ANDROID™, WINDOWS™ or BLACKBERRY™.

It will be appreciated by those skilled in the art that other variations of the embodiments described herein may also be practiced without departing from the scope of the invention. Other modifications are therefore possible.

In further aspects, the disclosure provides systems, devices, methods, and computer programming products, including non-transient machine-readable instruction sets, for use in implementing such methods and enabling the functionality described previously.

Although the disclosure has been described and illustrated in exemplary forms with a certain degree of particularity, it is noted that the description and illustrations have been made by way of example only. Numerous changes in the details of construction and combination and arrangement of parts and steps may be made. Accordingly, such changes are intended to be included in the invention, the scope of which is defined by the claims.

Except to the extent explicitly stated or inherent within the processes described, including any optional steps or components thereof, no required order, sequence, or combination is intended or implied. As will be will be understood by those skilled in the relevant arts, with respect to both processes and any systems, devices, etc., described herein, a wide range of variations is possible, and even advantageous, in various circumstances, without departing from the scope of the invention, which is to be limited only by the claims.

1. A system for monitoring healthcare of patients who are outside of a hospital environment, the system comprising:
an electronic alert data storage for storing a plurality of alert generation definitions, each of the alert generation definitions defining at least one condition for generating an alert;
a patient profile module configured to generate a patient profile data structure for a patient, the patient profile data structure comprising indicators of characteristics of the patient;
a patient monitoring module configured to:
receive monitoring data reflecting at least one of (i) physiological indicators of the patient; and (ii) indicators of medical supplies being ordered or consumed by the patient; and
generate an alert relating to patient healthcare by applying at least one of the plurality of alert generation definitions to the received monitoring data; and
an alert update module configured to:
receive population data reflective of healthcare outcomes for a patient population and monitoring data for the patient population;
generate at least one further alert generation definition upon processing the population data; and
update the electronic alert data storage with the at least one further alert generation definition.

2. The system of claim 1 wherein the population data further comprises data reflective of generated alerts for the patient population.

3. The system of claim 1, wherein said generating at least one further alert generation definition comprises training an artificial neural network using the population data.

4. The system of claim 1, further comprising:
an alert notification module configured to present a notification of the generated alert to a healthcare provider.

5. The system of claim 1, further comprising:
a network interface configured for interconnection with a communication network;
and wherein at least part of the monitoring data is received by way of the communication network.

6. (canceled)

7. The system of claim 1, wherein a machine learning technique is used to generate at least one further alert generation definition upon processing the population data.

8. (canceled)

9. The system of claim 1, wherein the alert update module is configured to generate the at least one further alert generation definition upon processing the population data by:
provisioning a neural network configured to refine the alert generation definitions for an alert using the population data;
applying one or more weights corresponding each of the at least one conditions for generating the alert, wherein an aggregate of the weights is used in determining whether an alert is triggered;
generating one or more predicted outcomes based on the alert rule;
providing the population data as an input into the neural network, the population data having various inputs and actual outcomes;
causing the neural network to iteratively process alert generation definitions based on the input population data, wherein the one or more weights corresponding to each of the at least one condition are varied, and on each iteration: using population data related to an individual in a population, determine whether each of the at least one conditions for generating the alert corresponds to a related actual outcome, if so, increase the weight corresponding to that condition, and if not, decrease the weight corresponding to that condition;
generating a further alert generation definition based on the one or more weights corresponding to each of the at least one condition following the use of the neural network to process iterations of alert generation definitions.

10. (canceled)

11. The system of claim 1, wherein the further alert generation definition is based on processed population data including at least both physiological indicators of other patients and indicators of medical supplies being ordered or consumed by other patients.

12. The system of claim 11, wherein the medical supplies include at least one of bandages, dialysate, dialysate bags, medical masks, gloves, tubing, catheters, antibiotics, hormones, and saline solutions.

13. The system of claim 9, wherein the one or more predicted outcomes include at least one of a fever, abdominal pain, cloudy effluent, vomiting, swollen appendages, slow inflow of dialysate, and slow outflow of dialysate.

14. A computer-implemented method of monitoring healthcare of patients who are outside of a hospital environment, the method comprising:
storing a plurality of alert generation definitions in electronic alert data storage, each of the alert generation definitions defining at least one condition for generating an alert;
generating, at at least one processor, a patient profile data structure for a patient, the patient profile data structure comprising indicators of characteristics of the patient;
receiving, at the at least one processor, monitoring data reflecting at least one of (i) physiological indicators of the patient; and (ii) indicators of medical supplies being ordered or consumed by the patient;
generating, at the at least one processor, an alert relating to patient healthcare by applying at least one of the plurality of alert generation definitions to the received monitoring data; and
receiving, at the at least one processor, population data reflective of healthcare outcomes for a patient population and monitoring data for the patient population;
generating, at the at least one processor, at least one further alert generation definition upon processing the population data; and
updating, at the at least one processor, the electronic alert data storage with the at least one further alert generation definition.

15. The method of claim 14, further comprising:
receiving, at the at least one processor, further monitoring data; and
generating, at the at least one processor, a further alert relating to patient healthcare by applying the at least one further alert generation definition to the received further monitoring data.

16. A system for facilitating management of chronic conditions in patient residences, the system comprising:
a patient profile module configured to generate a patient profile data structure for a patient, the patient profile data structure comprising:
indicators of a chronic condition of the patient, and
indicators of a treatment for the patient;
a patient monitoring module configured to receive monitoring data reflecting at least one of (i) physiological indicators of the patient; and (ii) indicators of medical supplies being ordered or consumed by the patient; and
a decision support module configured to:
process the patient profile data structure and the received monitoring data;
upon said processing, generate treatment suggestions for the patient; and
present the generated treatment suggestions to the patient.

17. (canceled)

18. The system of claim 16, further comprising:
a communications module configured to:
provide a communication interface for interaction between the patient and a health care provider.

19. The system of claim 16, wherein the communications module configured to:
match the patient with other patients for interaction therewith, the matching comprising processing at least one of the (i) the patient profile data structure and (ii) the received monitoring data; and
provide a communication interface for interaction between the patient and the other patients.

20. The system of claim 16, wherein the chronic condition of the patient includes renal failure.

21. The system of claim 20, wherein the medical supplies being ordered or consumed by the patient include at least one of face masks, gauze, bandages, dialysate, catheters, needles, and dialysate bags.

22. The system of claim 20, wherein the indicators of medical supplies being ordered or consumed by the patient are provided by one or more suppliers of the medical supplies.

23. The system of claim 22, wherein the indicators of medical supplies being ordered or consumed by the patient include logistics information related to the delivery of supplies to a patient's residence.

24. The system of claim 23, wherein the logistics information includes at least one of frequency of delivery, expected time of delivery, reliability of delivery, and lead-time for placing a supply order.

25. (canceled)
